(19) United States
(12) Patent Application Publication
(10) Pub. No.: US 2026/0287592 A1
(43) Pub. Date: Sep. 24, 2026

(54) ANALYSIS METHOD AND ANALYSIS SYSTEM

(71) Applicant: Konica Minolta, Inc., Tokyo (JP)

(72) Inventors: Naoko FURUSAWA, Hino-shi, Tokyo (JP); Hiroshi KITA, Hachioji-shi, Tokyo (JP); Naoya SAITO, Nishitama-gun, Tokyo (JP); Keiji KAJIWARA, Tsukuba-shi, Ibaraki (JP)

(21) Appl. No.: 19/168,465

(22) PCT Filed: Mar. 26, 2024
(86) PCT No.: PCT/JP2024/011835

§ 371 Date: Sep. 24, 2025

(30) Foreign Application Priority Data
Mar. 27, 2023 (JP) 2023-049165

(51) Int. Cl. G01N 33/58; C12Q 1/6816; G01N 33/543

(52) U.S. Cl. CPC G01N 33/582; C12Q 1/6816; G01N 33/54313

(57) ABSTRACT

Disclosed is an analysis method including: acquiring first information on expression of a specific protein that characterizes a function of a cell or a microorganism; acquiring second information on at least one of: a metabolite produced by the cell or the microorganism as a result of performing the function; or a composition constituting the cell or the microorganism; and, based on the first information and the second information, analyzing at least the function or a state of the cell or the microorganism.

### CROSS REFERENCE TO RELATED APPLICATIONS

This is the U.S. national stage of application No. PCT/JP2024/011835, filed on Mar. 26, 2024. Priority under 35 U.S.C. § 119 (a) and 35 U.S.C. § 365 (b) is claimed from Japanese Application No. 2023-049165 filed on Mar. 27, 2023, the disclosure of which is also incorporated herein by reference.

### TECHNICAL FIELD

The present invention relates to an analysis method and an analysis system. More particularly, the present invention relates to an analysis method and the like with improved accuracy.

### BACKGROUND ART

Recent developments in biotechnology, especially genetic engineering technology, have attracted attention to highly functionalized cells such as genetically modified cells. However, in order to industrially use highly functionalized cells in the future, there is a need for a technique of quantifying and analyzing the function and the state of the cells from the viewpoint of production technology and quality control.

In accordance with the central dogma, a cell synthesizes a protein from a DNA base sequence, which is the blueprint of the cell, through a translation process into mRNA. Then, as a result of the protein performing its function, a metabolite is produced. Therefore, by acquiring information on protein expression and/or information on a metabolite in a cell, the function or the state of the cell can be quantified and analyzed.

In the related art, as disclosed in Non-patent Documents 1, 2, and 3, attempts have been made to accurately grasp the state of cells by comprehensively analyzing metabolites of the cells.

### CITATION LIST

### Non-Patent Literature

Non-patent Document 1: Kusano, Miyako and Kazuki Saito. “Metaboromikusu no Hiraku Baiosaiensu no Atarashii Chihei (Metabolomics Opens New Horizons in Bioscience).”, Kagaku to Seibutsu (Chemistry and Biology), Japan Society for Bioscience, Biotechnology, and Agrochemistry, Vol. 43, No. 2, pp. 101-108.
Non-patent Document 2: Eiichiro Fukusaki. “Metaboromikusu Gijutsu Kaihatsu to Seimitsu Hyogengata eno Oyo (Development of Metabolomics Technology and its Application to Precise Phenotypic Analysis).”, Seibutsu Kogaku Gakkaishi (Journal of the Society for Biotechnology), The Society for Biotechnology, Japan, Vol. 94, No. 5, pp. 230-236.
Non-patent Document 3: Masahiro Sugimoto. “Metaboromu Kaiseki wo Mochiita Baiomaka Tansaku (Metabolomics-based Biomarker Discovery).”, The Journal of Tokyo Medical University, Tokyo Ika Daigaku Igakukai (Tokyo Medical University Medical Association), Vol. 76, No. 1, pp. 11-21.

### SUMMARY OF INVENTION

### Technical Problem

However, the mechanisms of the technologies disclosed in Non-patent Documents 1, 2, and 3 do not take into consideration the influence of specific proteins that greatly influence metabolites. Therefore, the accuracy of the technologies cannot be considered adequate.

The present invention has been made in consideration of the above-described problems and situations, and an object of the present invention is to provide an analysis method and an analysis system with improved accuracy.

### Solution to Problem

An analysis method comprising the steps of: acquiring first information on expression of a specific protein that characterizes a function of a cell or a microorganism; acquiring second information on at least one of: a metabolite produced by the cell or the microorganism as a result of performing the function; or a composition constituting the cell or the microorganism; and, based on the first information and the second information, analyzing at least the function or a state of the cell or the microorganism.

According to the above means of the present invention, it is possible to provide an analysis method and an analysis system with improved accuracy.

### BRIEF DESCRIPTION OF DRAWINGS

FIG. 1 This is a flowchart illustrating a conventional analysis method for a cell or the like.

FIG. 2 This is a flowchart illustrating a analysis method for a cell or the like according to the present invention.

FIG. 3 This is an explanatory diagram illustrating a mechanism in which an oligonucleotide probe interacts with a target substance to emit light.

FIG. 4 This is an explanatory diagram illustrating a method for producing an oligonucleotide probe.

FIG. 5 This is a flowchart illustrating an example of the analysis method.

### DESCRIPTION OF EMBODIMENTS

An analysis method according to the present invention includes the steps of: acquiring first information on expression of a specific protein that characterizes a function of a cell or a microorganism; acquiring second information on at least one of: a metabolite produced by the cell or the microorganism as a result of performing the function; or a composition constituting the cell or the microorganism; and, based on the first information and the second information, analyzing at least the function or a state of the cell or the microorganism.

This feature is a technical feature common to or corresponding to the following embodiments.

As an embodiment of the present invention, from the viewpoint of improving accuracy, it is preferable that the analysis method further includes the steps of: separating, from a first sample obtained by culturing the cell or the microorganism in a culture solution, the cultured cell or microorganism and a rest of the culture solution to respectively obtain a second sample and a third sample; crushing the cultured cell or microorganism to obtain a fourth sample; acquiring, from the second sample, the first information on the expression of the specific protein that characterizes the function of the cell or the microorganism; acquiring, from the third sample or the fourth sample, the second information on at least one of: the metabolite produced by the cell or the microorganism as a result of performing the function; or the composition constituting the cell or the microorganism; and, based on the first information and the second information, analyzing at least the function and the state of the cell or the microorganism.

As an embodiment of the present invention, from the viewpoint of simplicity of the steps it is preferable that at least one of the information on the expression of the protein, the information on the metabolite, or the information on the composition constituting the cell or the microorganism is obtained using a probe labeled with a luminescent substance.

As an embodiment of the present invention, from the viewpoint of high sensitivity, it is preferable that the step of acquiring the first information on the expression of the protein includes using the probe that includes the resin particle containing the luminescent dye, and the step of acquiring the second information on at least one of: the metabolite; or the composition constituting the cell or the microorganism includes using the oligonucleotide probe labeled with the luminescent substance.

An analysis system according to the present invention is an analysis system for a cell or a microorganism and is characterized by using the above-described analysis method.

Hereinafter, the present invention, constituent elements thereof, and modes and aspects for carrying out the present invention will be described in detail. In the present description, when two numbers are used to indicate a range of value before and after “to”, these numbers are included in the range as the lower limit value and the upper limit value.

Before explaining the details of the present invention, the background, the importance of the culture sensing technology, and the problems of the known technology will be described first.

### 1. Background

### (1) Inflection Point of Society

Against the backdrop of a deteriorating global environment and food shortages caused by population growth, the realization of a zero-carbon society has become an urgent need. The coronavirus pandemic has also brought medical issues to the forefront. To address these issues, the use of biotechnology is gaining attention, and governments around the world are taking action.

The U.S. bio-related industry initiative announced on Sep. 12, 2022, states that “Global industry is on the cusp of an industrial revolution powered by biotechnology.” It also states that the US will invest in biotechnology on a national scale. The Chinese government has designated biotechnology as its next key industry, investing more than 11 trillion yen in research and development.

In Japan, the “Bio-Strategy” compiled in 2019 sets the goal of realizing the world's most advanced bioeconomy society by 2030. The proposal states, “The combination of biotechnology (biology) with IT/AI technology is opening up new horizons for industry. This is truly a paradigm shift in industrial structure that deserves to be called the ‘fifth industrial revolution.’”

As a pillar of industry that drives the next generation of economic society, the bioindustry aims to spread and realize applications not only in the health and medical fields but also in the environmental, energy, materials, food and other fields. A “bioeconomic society” in which biotechnology supports the foundation of a wide range of industries is emerging globally.

### (2) Technical Background

Industrial development through biotechnology is progressing rapidly around the world in the fields of health, medicine, environment, energy, materials, and food. Behind this is the development of technology that can quickly and accurately elucidate the functions of living organisms at the genetic level through the fusion of IT/AI technology, which has been undergoing rapid technological innovation in recent years, and biotechnology. In addition, living organisms (cells) required for expressing such functions are designed on a computer. And indeed, it is becoming possible to form them as genetically modified cells at a level that can be utilized in industry. Industrial applications with cells are finally entering the practical stage.

### 2. Importance of Culture Sensing Technology

### (1) Importance of Culture Sensing Technology

Recent developments in biotechnology, particularly genetic engineering techniques, have made it possible to form highly functionalized cells such as genetically modified cells. The highly functionalized cells are becoming the key to the fifth industrial revolution, which leads to structural change, sophistication, and added value in various industrial fields.

Specifically, examples include regenerative medicine using iPS cell technology in the drug discovery and medical fields, genetically modified yeast in the chemical industry, and cultured meat in the food industry. Practical application and implementation of these technologies in industrialization requires culturing cells and controlling the synthesis of products that the cells produce with high accuracy as a production technology. In other words, a sensing technology for cell culture conditions, which supports production technology and quality control, plays an important role in ensuring quality and high productivity.

### (2) Importance of Analysis in Cell Culture Sensing Technology

What, then, is required as a sensing technology for cell culture? We focused on proteins expressed by DNA transfection, metabolites produced by the functions of the proteins, and compositions constituting cells.

In accordance with the central dogma, a cell synthesizes a protein from a DNA base sequence, which is the blueprint of the cell, through a translation process into mRNA. Then, as a result of the protein performing its function, a metabolite is produced.

In a genetically modified cell, a recombinant protein for enhancing the production amount and/or the quality of a target substance is expressed. As a result of recombinant gene transfer, the recombinant protein is expressed in the cell. It is precisely this protein that produces a product in the cell.

Further, it is known that the expression amount of a recombinant protein varies depending on culture conditions. It is also known that metabolite production is affected by culture conditions even in cells expressing the same amount of recombinant protein. From this point of view, accurate culture control cannot be achieved by measuring only proteins or only metabolites.

That is, in the production technology using biotechnology, stabilization of the culture process and maximization of process efficiency are required. In addition, precise control of culture conditions is required for a production system of cultured cells. To this end, it is necessary to measure and analyze both the proteins that serve as intermediate descriptors linking DNA to metabolite production and the metabolites that serve as the final products.

An example of the production technology using biotechnology is a technology for producing bioplastics using strains. With this technology, the reaction process of a vegetable oil serving as a raw material is controlled by three main enzymes in cells. These three proteins are known in advance and play important roles in controlling the reaction efficiency and reaction process.

Another example of the production technique using biotechnology is a technique for expressing an enzyme such as cellulase on the surface of yeast or a microorganism by cell surface engineering. The enzyme on the cell surface breaks down cellulose raw materials to synthesize bioethanol and the like. In this case, the amount of protein expressed on the cell surface by gene transfer becomes an important factor in determining the reaction efficiency.

The development of next-generation sequencers has led to precise evaluation of cell culture genes. However, even if a DNA sequence artificially modified by genetic engineering is introduced into a gene of a cell as intended, various processes exist before the gene is actually translated to produce the desired substance (metabolite).

Even if the modified DNA has been introduced as intended, it is not possible to evaluate from genetic analysis whether the protein synthesized from the blueprint of the introduced DNA is “expressed” as intended in the cell, as well as the level of expression. Even if a protein has been expressed in a cell, it is not possible to evaluate from the protein expression amount whether the protein is “functioning” as intended. Therefore, to accurately evaluate whether a DNA-modified cell is functioning as designed, it is necessary to evaluate not only the DNA sequence after modification, but also the expression state of the protein in the cell, and a metabolite produced by the protein as a result of performing its function. Furthermore, in some cases, it is necessary to evaluate constituent compositions as a whole.

In the analysis of a complex system including thousands to tens of thousands of compounds, such as a metabolite and a constituent composition, a (exhaustive) method in which all the compounds are targeted for measurement without specifying an analyte has been investigated. In the method in which all the compounds are targeted for measurement, for example, obtained data are clustered by machine learning to extract and analyze desired information. Furthermore, in addition to the exhaustive analysis of a metabolite, measurement of the abundance of a targeted protein can also be used for machine learning. Proteins are factors that greatly affect the production of metabolites.

The analysis method will be considered from the viewpoint of the size of the object to be measured. Sensing technologies in various industrial applications preferably select a measurement method according to the size of the target object. When a plurality of target objects having different sizes are simultaneously evaluated, multi-scale measurement in which a plurality of measurement methods is combined is effective.

In an exhaustive analysis of a metabolite and/or a constituent composition, the target compounds are low molecular weight compounds in the range of 1 to several nm, which are likely to interact with DNA probes of similar size. On the other hand, a protein is larger than the low-molecular-weight compounds, with a size of 10 nm or more. For detecting compounds of this size, a detection method using nanoparticles that have a size of several tens of nanometers and give a strong signal is suitable.

As described above, an effective probe is different depending on the size of the target object. By combining probes suitable for the target object and performing multi-scale measurement, high-sensitivity detection becomes possible. For example, the use of a fluorescent dye as a probe is advantageous in that the probe has high sensitivity and a detection device is inexpensive. Furthermore, the preparation of an experimental sample is relatively easy, and the culturing step can be controlled at low cost and with little labor.

### (3) Problems with Current Methods of Analysis

A protein can be detected using an antibody that binds to the target protein. However, there are problems with a method that uses a fluorescent dye as a labeling material to detect a bound antibody. A single dye molecule has low luminance, and high-sensitivity quantification cannot be performed due to photofading properties of the dye molecule.

A metabolite and a constituent composition are composed of low molecular weight compounds such as amino acids, lipids, and carbohydrates of many kinds, which are said to number in the thousands to tens of thousands. Although the compounds serve as a very important indicator from the viewpoint of reflecting the state of a cell at any given time, there is no method for exhaustively analyzing the compounds as a whole. Currently, only a portion of the metabolite and/or constituent composition is analyzed by a mass spectrometer or the like.

Due to these problems, further technical improvement is required for the analysis of proteins and their metabolites and constituent compositions.

### (4) Importance of Analysis in Each Technical Field

### (4.1) Importance in the Drug Discovery Field

Examples of genetically modified cell technologies in the field of drug discovery include the practical application of CAR-T therapy in cancer treatment, in which immune cells in the patient's blood are genetically modified. Other examples include drug discovery and regenerative medicine using iPS cells.

In the case of iPS cells, undifferentiated cells obtained by genetic engineering are induced to differentiate into cells of various organs in the culture process, and the differentiated cells are used in regenerative medicine and drug discovery evaluation. For efficient differentiation into cells of uniform quality, it is important that the differentiated cells express organ proteins as intended, and that the cells function normally as organ cells. For high-quality and precise cell culture control and process control, analysis of protein expression amounts and metabolites in culture media is required.

### (4.2) Importance in the Chemical Industry

In the chemical industry, synthetic biology has been attracting attention due to the advent of a zero-carbon society. In order to prevent global environmental degradation and global warming, governments around the world, including Japan, the United States, and China, have launched a series of policies to promote their initiatives. As part of this effort, examples include the synthesis of carbon-neutral polymers using biomass and CO₂ as raw materials, and the efficient production of fats and oils, biofuels, and other products using biomass as a raw material.

To realize these initiatives, technological development has begun for biomanufacturing using genetically engineered yeast and microorganisms as genetically engineered smart cells. The expression and expression levels of proteins produced in cells by gene recombination are the very function that produces products and are directly linked to quality control and production efficiency. In addition, analysis of metabolites is the quality of the products itself and is an indispensable analysis item.

### (4.3) Importance in the Food Industry

Against the backdrop of a rapidly shrinking working population and global climate change, the era of easy access to edible meat is beginning to show signs of decline. Therefore, cultured meat is attracting attention as a means for obtaining animal protein. Cultured meat is produced, for example, by isolating bovine skeletal muscle-derived cells from cattle, differentiating the cells into muscle fibers, and culturing the cells on a collagen scaffold structure. That is, in controlling cell differentiation and culture, the expression of proteins that become muscle cells themself is important for quality control. Analysis of metabolites that reflect the states of the cells in culture is important for production control.

### 3. Problems of Known Technology

### (1) Analysis of Protein

Examples of an analysis method for a protein that is a polymer compound include an analysis method using a mass spectrometer and an analysis method utilizing a fluorescent dye or chemiluminescence.

In the analysis of a protein by mass spectrometry, pretreatment is complicated, and sensitivity is low. On the other hand, the method in which an antibody is detected and analyzed by using a fluorescently labeled substance (fluorescently labeled probe) is simpler than mass spectrometry. However, fluorescent dyes have low luminance per molecule and, furthermore, fade when exposed to observation light. Therefore, in the analysis using the fluorescently labeled substance, there has been a limit to high-sensitivity quantification. In addition, since chemiluminescence emits light by a chemical reaction and its light emission intensity is affected by the environment, chemiluminescence is not suitable for quantification, and there has been a limit to high-sensitivity quantification as in the case of the fluorescent dye.

### (2) Analysis of Metabolites

Metabolites are compounds produced from biological activities, such as peptides, sugars, and lipids and include thousands to tens of thousands of types. Since the composition and ratio of metabolites reflect the current state of the cell, metabolites can be said to be phenotypes of life phenomena.

However, although examples of an analysis method for some of the metabolites include an analysis method using a mass spectrometer, there is currently no method for exhaustively analyzing all metabolites. With recent advances in machine learning, a non-targeting method in which all signals are analyzed using a mass spectrometer without specifying a target has attracted attention. However, due to various restrictions such as pretreatment and column selection, the analysis of all metabolites has not been achieved yet and is insufficient.

### (3) Analysis of Compounds Constituting Cell

The compounds constituting a cell are water, inorganic ions, and carbon-containing organic molecules. Among these, specific examples of the inorganic ions include sodium ions (Na⁺), potassium ions (K⁺), magnesium ions (Mg²⁺), calcium ions (Ca²⁺), hydrogen phosphate ions (HPO₄²⁻), chloride ions (Cl⁻), and hydrogen carbonate ions (HCO₃⁻). Examples of the carbon-containing organic molecules include carbohydrates, lipids, nucleic acids, and proteins. Specifically, examples of the carbohydrates include monosaccharides and polysaccharides. Examples of the lipids include lipids, fatty acids, phospholipids, glycolipids, and cholesterol. Examples of the nucleic acids include deoxyribonucleic acid and ribonucleic acid. A protein is a polymer of 20 different amino acids and is a compound having various three-dimensional structures.

The substances constituting a cell are thus diverse. The entirety of proteins constituting a cell reflects the cell itself, which is literally a living organism. Furthermore, by adding information on metabolites, which are the results of life activities, to the compounds constituting the cell, it is possible to acquire a wider range of information from which the state of the cell can be grasped, thereby increasing the accuracy of analysis.

### 4. Summary of the Present Invention

The present invention is a new sensing technology that combines high-accuracy quantification of proteins, which is a deductive method, with exhaustive measurement of metabolites or constituent compositions, which is an inductive method.

The present invention quantifies a specific protein expressed by DNA transfected by genetic modification or the like in cells, cancer cells, and the like. Then, substances around those cells, such as a culture solution and serum that contain metabolites produced by the cells, are exhaustively analyzed without specifying a target metabolite. Furthermore, compounds constituting those cells are exhaustively analyzed without specifying a target compound. By combining the results of protein quantification with the results of metabolite or constituent composition analysis, the states of the cells can be analyzed with high accuracy.

The present invention and conventional methods are compared in Tables I and II below.

TABLE I

| | ANALYSIS TARGET OBJECT | ACCURACY |
|---|---|---|
| PRIOR ART | “SPECIFIC PROTEIN” ONLY | HAVING LOW ACCURACY AND |
| 1 | | INSUFFICIENT FOR QUALITY CHECK, |
| | | PROCESS CONTROL, AND THE LIKE |
| PRIOR ART | “EXHAUSTIVE METABOLITES” ONLY | HAVING LOW ACCURACY AND |
| 2 | | INSUFFICIENT FOR QUALITY CHECK, |
| | | PROCESS CONTROL, AND THE LIKE |
| PRESENT | MEASURE BOTH “SPECIFIC PROTEIN” | HAVING HIGH ACCURACY AND |
| INVENTION | AND “EXHAUSTIVE METABOLITES” | SUFFICIENT FOR QUALITY CHECK, |
| 1 | | PROCESS CONTROL, AND THE LIKE |
| PRESENT | MEASURE BOTH “SPECIFIC PROTEIN” | HAVING HIGH ACCURACY AND |
| INVENTION | AND “EXHAUSTIVE COMPOSITIONS | SUFFICIENT FOR QUALITY CHECK, |
| 2 | CONSTITUTING CELLS” | PROCESS CONTROL, AND THE LIKE |
| PRESENT | MEASURE BOTH “SPECIFIC PROTEIN” | HAVING PARTICULARLY HIGH |
| INVENTION | AND “EXHAUSTIVE METABOLITES” | ACCURACY AND SUFFICIENT FOR |
| 3 | AND “EXHAUSTIVE COMPOSITIONS | QUALITY CHECK, PROCESS CONTROL, |
| | CONSTITUTING CELLS” | AND THE LIKE, AND PARTICULARLY |
| | | PREFERABLE |

TABLE II

| | | ANALYSIS METHOD | FEATURES |
|---|---|---|---|
| ANALYSIS OF | PRIOR | METHOD FOR DETECTING | SENSITIVITY IS LOW |
| “SPECIFIC | ART | PROTEIN USING SINGLE | BECAUSE ONE MOLECULE OF |
| PROTEIN” | | FLUORESCENT DYE AND | FLUORESCENT DYE |
| | | ANALYZING IMAGE | CORRESPONDS TO ONE |
| | | | MOLECULE OF PROTEIN |
| | | METHOD OF QUANTIFICATION | PROCESS IS COMPLICATED |
| | | USING MASS SPECTROMETER | AND SENSITIVITY IS LOW |
| | PRESENT | METHOD FOR DETECTING | SENSITIVITY IS HIGH |
| | INVENTION | PROTEIN USING | BECAUSE MULTIPLE |
| | | HIGH-BRIGHTNESS | MOLECULES OF |
| | | FLUORESCENT | FLUORESCENT DYE |
| | | NANOPARTICLES AND | CORRESPOND TO ONE |
| | | ANALYZING IMAGE | MOLECULE OF PROTEIN |
| | | | PROCESS IS SIMPLE |
| EXHAUSTIVE | PRIOR | METHOD OF QUANTIFICATION | PROCESS IS COMPLICATED |
| ANALYSIS OF | ART | USING MASS SPECTROMETER | TARGET IS NOT ALL |
| “METABOLITES” | | | METABOLITES BUT LIMITED |
| AND “COMPOSITIONS | | | TO SOME |
| CONSTITUTING CELLS” | PRESENT | METHOD FOR DETECTING | TARGET IS ALL METABOLITES |
| | INVENTION | METABOLITES USING | PROCESS IS SIMPLE |
| | 1 | OLIGONUCLEOTIDE PROBE | |
| | | AND ANALYZING SIGNALS | |
| | PRESENT | METHOD FOR DETECTING | TARGET IS ALL COMPOUNDS |
| | INVENTION | COMPOSITIONS | CONSTITUTING CELLS |
| | 2 | CONSTITUTING CELLS USING | PROCESS IS SIMPLE |
| | | OLIGONUCLEOTIDE PROBE | |
| | | AND ANALYZING SIGNALS | |
| | PRESENT | METHOD FOR DETECTING | TARGET IS ALL METABOLITES |
| | INVENTION | METABOLITES AND | AND ALL COMPOUNDS |
| | 3 | COMPOSITIONS | CONSTITUTING CELLS |
| | | CONSTITUTING CELLS USING | PROCESS IS SIMPLE |
| | | OLIGONUCLEOTIDE PROBE | |
| | | AND ANALYZING SIGNALS | |

### 5. Details of Analysis Method of the Present Invention

The analysis method according to the present invention includes the steps of: acquiring the first information on the expression of a specific protein that characterizes a function of a cell or a microorganism; acquiring the second information on at least one of: a metabolite produced by the cell or the microorganism as a result of performing the function; or a composition constituting the cell or the microorganism; and, based on the first information and the second information, analyzing at least the function or the state of the cell or the microorganism.

The term “composition constituting the cell or the microorganism” refers to an aggregate of compounds constituting the cell or the microorganism.

The compounds constituting the cell are water, inorganic ions, and carbon-containing organic molecules. Among these, specific examples of the inorganic ions include sodium ions (Na⁺), potassium ions (K⁺), magnesium ions (Mg²⁺), calcium ions (Ca²⁺), hydrogen phosphate ions (HPO₄²⁻), chloride ions (Cl⁻), and hydrogen carbonate ions (HCO₃⁻). Examples of the carbon-containing organic molecules include carbohydrates, lipids, nucleic acids, and proteins. Specifically, examples of the carbohydrates include monosaccharides and polysaccharides. Examples of the lipids include lipids, fatty acids, phospholipids, glycolipids, and cholesterol. Examples of the nucleic acids include deoxyribonucleic acid and ribonucleic acid. A protein is a polymer of 20 different amino acids and is a compound having various three-dimensional structures.

In detail, the analysis method according to the present invention acquires each of the following two pieces of information and performs comprehensive analysis based on the two pieces of information. By comprehensively analyzing the two pieces of information, the cell or the microorganism can be analyzed with higher accuracy.

### Pattern 1

(A) information on the expression of a specific protein that characterizes a function of the cell or the microorganism
(B) information on a metabolite produced by the cell or the microorganism as a result of performing the function

Hereinafter, the information (A) is also simply referred to as “protein information”, and the information (B) is also simply referred to as “metabolite information”. In addition, in the present specification, “a/the cell or the like” has the same meaning as “a/the cell or microorganism”.

### Pattern 2

(A) information on the expression of a specific protein that characterizes a function of the cell or the microorganism
(C) information on the composition constituting the cell or the microorganism

Hereinafter, the information (C) is also simply referred to as “constituent composition information”.

### Pattern 3

(A) information on the expression of a specific protein that characterizes a function of the cell or the microorganism
(D) information on the metabolite produced by the cell or the microorganism as a result of performing the function and the composition constituting the cell or the microorganism

Hereinafter, the information (D) is also simply referred to as “metabolite and constituent composition information”.

Test objects used to acquire each of the above two pieces of information are the second, third and fourth samples collected from the same sample (first sample) containing the cell or the microorganism, and the metabolite. Either one of the third sample and the fourth sample may be used, or both may be used.

In the present invention, the term “test object” refers to an individual such as a cell, microorganism, or metabolite that is the target of measurement or analysis.

The “first sample” is separated or collected from the target object and contains the cell or the microorganism, and the metabolite.

The “second sample” is collected from the first sample and contains the cell or the microorganism. The second sample may be composed of the cell or the microorganism only or may contain other components.

The “third sample” is collected from the first sample and contains the metabolite. The third sample may be composed of the metabolite alone or may contain other components.

The “fourth sample” is obtained by crushing the collected cell or the microorganism. The cell or the microorganism may be separated or collected from the target object or may be collected from the first sample.

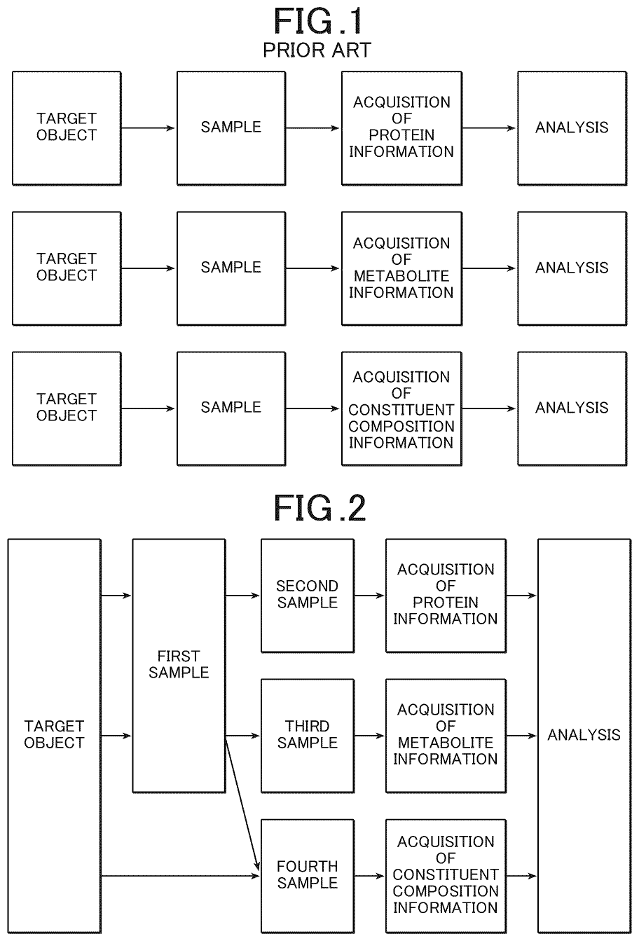

FIG. 1 is a flowchart illustrating a conventional analysis method for a cell or the like. In this method, only protein information, metabolite information, or constituent composition information is acquired from the target object to analyze the cell or the like.

A conventional analysis method for a cell or the like has included separately acquiring and analyzing protein information, metabolite information, and constituent composition information. However, as described above, in recent years, there has been a need to analyze the functions and states of cells or the like in greater detail, and conventional methods have had limitations regarding the accuracy of analysis.

FIG. 2 is a flowchart illustrating the analysis method for a cell or the like according to the present invention. In the analysis method according to the present invention, the first sample is collected from the target object. Furthermore, the second sample (sample for acquiring the protein information), the third sample (sample for acquiring the metabolite information), and the fourth sample (sample for acquiring the constituent composition information) are prepared. Then, the protein information, and the metabolite information or the constituent composition information are acquired, and the acquired information is comprehensively analyzed.

In the analysis method according to the present invention, the protein information, and the metabolite information or the constituent composition information are comprehensively analyzed. Thus, it is considered that the accuracy of analysis is improved as compared with analysis from the protein information only, the metabolite information only, or the constituent composition information only. Furthermore, the sample for acquiring the protein information and the sample for acquiring the metabolite information are prepared from the same sample. Thus, the protein information and the metabolite information can be acquired simultaneously for cells or the like that reflect the same growth conditions at a specific time point and under specific conditions from the growth period to sampling. As a result, it is considered that the accuracy of analysis is improved.

### (1) APPLICATION EXAMPLES

Application examples of the present invention will be described.

In the medical field, examples include iPS cells and mesenchymal stem cells (MSC). In these cells, differentiation may not proceed as expected depending on culture conditions and the like. By applying the present invention in cell culture, it is considered that quality control and process control of cells can be performed with high accuracy.

In the chemical field, the environmental field, the energy field, and the like, examples include genetically modified plants. Bioplastics and biofuels using these plants have been commercialized. In cells with recombinant genes, functions may not be performed as expected depending on culture conditions and the like. By applying the present invention in cell culture, it is considered that quality control and process control of cells can be performed with high accuracy.

In the food sector, examples include cultured meat. In cultured meat, stem cells are differentiated into muscle cells to form colonies. In cells contained in the cultured meat, the colony formation may not proceed as expected depending on culture conditions and the like. By applying the present invention in cell culture, it is considered that quality control and process control of cells can be performed with high accuracy.

All the above three examples are in the technical field of culturing cells industrially. Then, by applying the present invention, it is possible to confirm the function and state of the target cell by comprehensively analyzing the protein information, and the metabolite information or the constituent composition information.

### (2) ANALYSIS TARGETS

### (Cell and Microorganism)

The type of the cell and the type of the microorganism are not particularly limited. Examples of the cell include animal cells, insect cells, and plant cells. Examples of the microorganism include archaea and bacteria.

Animal cells are broadly classified into cells derived from animals belonging to the vertebrate phylum and cells derived from invertebrates (animals other than animals belonging to the vertebrate phylum). The origin of the animal cells is not particularly limited.

Among them, the animal cells are preferably derived from animals belonging to the vertebrate phylum. The vertebrate phylum includes the agnatha superclass and the gnathostomata superclass. The gnathostomata superclass includes the mammal class, bird class, amphibian class, reptile class, and others. The animal cells are preferably derived from animals belonging to the mammal class known as mammals. The mammals are not particularly limited, and examples thereof include mice, rats, humans, monkeys, pigs, dogs, cats, sheep, and goats.

In particular, the animal cells are preferably Chinese hamster ovary tissue-derived cells (CHO cells), African green monkey kidney-derived established cell lines (Vero cells), canine kidney tubular epithelial cell-derived cell lines (MDCK cells), or human liver cancer tissue-derived established cell lines (huGK-14).

The origin of the plant cells is not particularly limited. Plant cells that include moss plants, fern plants, and seed plants are targeted.

Plants from which seed plant cells are derived include both monocotyledons and dicotyledons. Examples of the monocotyledons include orchidaceous plants, gramineous plants (rice, corn, barley, wheat, sorghum, and the like), and Cyperaceae plants. Examples of the dicotyledons include plants belonging to many subclasses, such as Asteridae, Magnoliidae, and Rosidae.

Algae are cell-derived organisms. Algae include different groups such as: cyanobacteria (blue-green algae), which are eubacteria; eukaryotes that are unicellular organisms (diatoms, yellow-green algae, dinoflagellates, and the like); and seaweeds (red algae, brown algae, green algae), which are multicellular organisms.

The archaea and the bacteria are not particularly limited. Examples of the archaea include methanogens, highly halophilic bacteria, thermophiles, and hyperthermophiles. Examples of the bacteria include yeast, lactic acid bacteria, Escherichia coli, Bacillus subtilis, and cyanobacteria.

The type of the animal cells or the plant cells is not particularly limited, but among them, pluripotent stem cells, tissue stem cells, somatic cells, or germ cells are preferable.

In the present invention, the term “pluripotent stem cells” refers to stem cells having the ability to differentiate into cells of any tissue (differentiation pluripotency). Examples of the pluripotent stem cells include, but are not particularly limited to, embryonic stem cells (ES cells), induced pluripotent stem cells (iPS cells), embryonic germ stem cells (EG cells), and germ stem cells (GS cells).

Among them, the pluripotent stem cells are preferably ES cells or iPS cells, and more preferably iPS cells from the viewpoint of having no ethical problem. Any known pluripotent stem cells can be used. For example, the pluripotent stem cells described in WO 2009/123349 (PCT/JP2009/057041) can be used.

The term “tissue stem cells” refers to stem cells that have the ability to differentiate into various cell types (differentiation pluripotency), although the differentiable cell lineage of the tissue stem cells is restricted to a specific tissue. For example, hematopoietic stem cells in the bone marrow give rise to blood cells, and neural stem cells differentiate into nerve cells. In addition, there are various types of tissue stem cells, such as liver stem cells that form the liver and skin stem cells that form skin tissue. Among them, the tissue stem cells are preferably mesenchymal stem cells, liver stem cells, pancreatic stem cells, neural stem cells, skin stem cells, or hematopoietic stem cells.

The term “somatic cells” refers to cells other than germ cells, among cells constituting a multicellular organism. In sexual reproduction, somatic cells are not passed on to the next generation. Examples of the somatic cells include hepatocytes, pancreatic cells, muscle cells, osteocytes, osteoblasts, osteoclasts, chondrocytes, adipocytes, skin cells, fibroblasts, pancreatic cells, renal cells, and lung cells. Other examples of the somatic cells include blood cells such as lymphocytes, erythrocytes, leukocytes, monocytes, macrophages and megakaryocytes.

The term “germ cells” refers to cells that play a role in transmitting genetic information to the next generation in reproduction. Examples of the germ cells include: gametes for sexual reproduction, i.e., ovums, egg cells, sperms, and sperm cells; and spores for asexual reproduction.

Other examples of the cells include sarcoma cells, established cell lines, and transformed cells.

“Sarcomas” are cancers that develop in connective tissue cells derived from non-epithelial cells such as bone, cartilage, fat, muscle, and blood. Examples of the sarcomas include soft tissue sarcomas and malignant bone tumors. The sarcoma cells are cells derived from sarcomas.

The term “established cell lines” refers to cultured cells that have been maintained outside the body for a long period of time, have attained certain stable properties, and can be semi-permanently passage-cultured. Examples of the established cell lines include PC12 cells (derived from rat adrenal medulla), CHO cells (derived from Chinese hamster ovary), HEK293 cells (derived from human embryonic kidney), HL-60 cells (derived from human leukocyte cells), HeLa cells (derived from human cervical cancer), Vero cells (derived from African green monkey kidney epithelial cells), and MDCK cells (derived from canine kidney tubular epithelial cells). As the established cell lines, there are cell lines derived from various tissues of various species including humans.

The term “transformed cells” refers to cells into which nucleic acids (DNA or the like) have been introduced from the outside of the cells to change the genetic properties.

### (Metabolite)

The term “metabolite” refers to a substance produced by metabolism, i.e., a growth by-product, or a substance that needs to participate in a particular metabolic process. The metabolite includes the starting material (e.g., glucose), intermediate (e.g., acetyl-CoA) and end product (e.g., n-butanol) of metabolism. The metabolite is not particularly limited, and examples thereof include biosurfactants, enzymes, acids, solvents, gases, alcohols, proteins, vitamins, minerals, trace elements, amino acids and polymers.

The “sample containing the cell or the like and the metabolite” may be a culture solution containing the cell or the like obtained in the culturing step of the cell or the like described above.

In one aspect, the analysis method according to the present invention aims to know the function and state of the cultured cell by analyzing the “sample containing the cell or the like and the metabolite” obtained in the culturing step.

### (3) SAMPLE CONTAINING CELL OR MICROORGANISM, AND METABOLITE

The target of the analysis method according to the present invention is the sample containing the cell or the microorganism, and the metabolite.

The sample according to the present invention is not particularly limited as long as the sample contains the cell or the like, and the metabolite.

The sample containing the cell or the like, and the metabolite may be a suspension of cells containing primary cells or established cell lines. A known method can be used as a method for preparing these cells and a sample containing the cells.

The “primary cells” refers to cells cultured by first seeding tissues or cells collected from a living organism. In general, the term refers to cells in culture prior to passaging, and in many cases, it is not a single cell but a mixture of various cells.

Cells obtained by passaging the primary cells are referred to as “passaged cells”. In addition, a series of cells that maintain their proliferative capacity through passaging are referred to as “established cell lines”. Examples of the established cell lines include PC12 cells (derived from rat adrenal medulla), CHO cells (derived from Chinese hamster ovary), HEK293 cells (derived from human embryonic kidney), HL-60 cells (derived from human leukocyte cells), HeLa cells (derived from human cervical cancer), Vero cells (derived from African green monkey kidney epithelial cells), MDCK cells (derived from canine kidney tubular epithelial cells), and HepG2 cells (derived from human hepatic carcinoma) 6. Example of Analysis Method

Hereinafter, an example of the analysis method according to the present invention will be described. However, the present invention is not limited thereto.

As an example of the analysis method according to the present invention, it is preferable to include the following steps.
(1) Step of separating, from the first sample obtained by culturing cells or microorganisms in a culture solution, the cultured cells or microorganisms and the rest of the culture solution to respectively obtain the second sample and the third sample
(2) Step of crushing the cultured cells or microorganisms to obtain the fourth sample
(3) Step of acquiring, from the second sample, the first information on the expression of a specific protein that characterizes a function of the cells or the microorganisms
(4) Step of acquiring, from the third sample or the fourth sample, the second information on at least one of metabolites produced by the cells or the microorganisms as a result of performing the function, or compositions constituting the cells or the microorganisms
(5) Step of analyzing at least the function or the state of the cells or the microorganisms based on the first information and the second information

Each of the steps will be described.

### (1) Step of Separating, from the First Sample Obtained by Culturing Cells or Microorganisms in a Culture Solution, the Cultured Cells or Microorganisms and the Rest of the Culture Solution to Respectively Obtain the Second Sample and the Third Sample

Under an environment in which the cells or the like is cultured, a portion of the culture solution containing the cells or the like is collected and used as the first sample. Then, the cultured cells or the like and the rest of the culture solution are separated to prepare the second sample and the third sample, respectively. The culture conditions are not particularly limited, and known cultured cells can be used.

In the present invention, the term “culture” refers to maintaining or proliferating cells or the like under artificially controlled suitable environmental conditions. The culturing step can be performed using a suitable culture medium and culture conditions known in the art. Such a culturing step can be easily adjusted by those skilled in the art, depending on the cell or the like. Specifically, the culture may be, but is not limited to, batch, continuous, or feeding culture.

In addition, the term “culture solution” refers to a liquid culture medium. In the step of culturing the cells, the culture solution contains metabolites produced by proteins as a result of performing their functions.

The term “culture medium” refers to a substance obtained by mixing, as its main component, nutritional substances necessary for culturing cells or the like. The culture medium supplies nutritional substances, such as water, which are essential for the survival and growth of cells or the like, as well as growth factors. The culture medium and other culture conditions used for culturing the cells or the like according to the present invention are not particularly limited as long as they are used for culturing general cells and the like. For example, the cells or the like can be cultured in a general culture medium containing a suitable carbon source, nitrogen source, phosphorus source, inorganic compound, amino acid, vitamin, or the like under aerobic conditions with adjustment of temperature, pH, and the like.

### (2) Step of Crushing the Cultured Cells or Microorganisms to Obtain the Fourth Sample

Under an environment in which the cells or the like are cultured, a portion of the cultured cells are collected and crushed to prepare the fourth sample.

For example, the fourth sample can be prepared by the following method.

A portion of the cultured cells are collected. Next, the collected cells are washed with phosphate-buffered saline (PBS) by centrifugation. Then, the washed cells are homogenized using an ultrasound disperser. A required amount of RIRA buffer (manufactured by Nacalai Tesque, Inc.) is added to the cells to prepare the sample (fourth sample) of the crushed cell liquid.

### (3) Step of Acquiring, from the Second Sample, the First Information on the Expression of a Specific Protein that Characterizes a Function of the Cells or the Microorganisms

As a method for acquiring the first information on the expression of the protein in the cells or the like, a known method can be used. From the viewpoint of high accuracy, it is preferable to use a probe labeled with a luminescent substance in the present invention.

In the present invention, the term “probe” refers to a substance that is labeled by some means and is used for detecting a protein and/or a metabolite.

Hereinafter, a method using a probe labeled with a fluorescent dye (fluorescent immunostaining method) will be described. In the present specification, the “probe labeled with a fluorescent dye” is also referred to simply as a “fluorescently labeled probe”.

From the viewpoint of sensitivity, the probe labeled with a luminescent substance is preferably a probe that includes resin particles containing the fluorescent dye (high-brightness fluorescent nanoparticles).

Examples of the probe labeled with a luminescent substance include, in addition to the high-brightness fluorescent nanoparticles, semiconductor nanoparticles as phosphors. In the semiconductor nanoparticles, semiconductors are used as the phosphors in place of the fluorescent dye in the high-brightness fluorescent nanoparticles described later.

### (3.1) Fluorescent Immunostaining Method

In the “fluorescent immunostaining method,” the specific protein in the cells or the like is labeled with the probe containing a fluorescent dye by means of an antibody to prepare a sample. Then, excitation light is applied to the sample. An image produced by fluorescence emitted from the molecules of the labeled protein is observed using, for example, a fluorescence microscope to observe the expression state of the specific protein in the cells or the like.

The fluorescent immunostaining method may be a direct method or an indirect method.

In the direct method, a primary antibody is specifically bound or immobilized to the specific protein (antigen). The primary antibody is then labeled with the probe containing a fluorescent dye.

On the other hand, in the indirect method, a primary antibody is specifically bound or immobilized to the specific protein (antigen). Next, a secondary antibody is specifically bound to the bound or immobilized primary antibody. Then, the secondary antibody is labeled with the probe containing a fluorescent dye.

From the viewpoint of ease of obtaining antibodies, the indirect method is preferred.

The protein (antigen) is not particularly limited as long as it is a protein to which the fluorescent immunostaining method can be applied.

### (3.1.1) Primary Antibody

The primary antibody is an antibody that recognizes an epitope unique to the antigen. The term “epitope” refers to a specific structural unit of an antigen that is recognized and bound by an antibody.

The primary antibody may be a free primary antibody.

When a polyclonal antibody is used as the primary antibody, a general polyclonal antibody prepared by using a specific sequence portion containing an epitope of the antigen as an immunizing antigen can be used. Not limited to this, for example, a mixture of two or more monoclonal antibodies may be used as a substitute (equivalent) for the polyclonal antibody. In this case, monoclonal antibodies that specifically bind to different respective epitopes are preferably combined.

The primary antibody does not need to be a full-length antibody, as is the case with a natural antibody, so long as the secondary antibody can bind to the primary antibody while the primary antigen is bound to the antigen. The primary antibody may be a fragment of an antibody or a derivative of an antibody. That is, in the present specification, the term “antibody” includes not only a full-length antibody but also a derivative such as an antibody fragment, a chimeric antibody (e.g., humanized antibody), and a multifunctional antibody.

Note that the type of animal that produces the primary antibody (immunized animal) is not particularly limited and can be selected from mouse, rat, guinea pig, rabbit, goat, sheep, and the like.

Preferably, the primary antibody is an IgG antibody. For example, when PD-L1 is used as the antigen, an anti-PD-L1 antibody is preferably used as the IgG antibody. When HER2 is used as the antigen, an anti-HER2 antibody is preferably used as the IgG antibody.

### (3.1.2) Secondary Antibody

The secondary antibody specifically recognizes an unreacted portion (Fc, F (ab), or F(ab′)) of the antigen of the primary antibody immobilized to the antigen. The secondary antibody then binds to a portion or all of the primary antibody. The secondary antibody does not bind to the antigen. The secondary antibody is, or will be in the future, bound to the fluorescently labeled probe.

Preferably, the secondary antibody is an IgG antibody. The secondary antibody is usually produced in a different immune animal than the primary antibody and is produced to recognize an antibody (e.g., Fc region) of the animal species of the primary antibody. The type of animal that produces the primary antibody and/or the secondary antibody (immunized animal) is not particularly limited, and can be selected from mouse, rat, guinea pig, rabbit, goat, sheep, and the like.

### (3.1.3) Binding of Secondary Antibody to Fluorescently Labeled Probe

The secondary antibody and the fluorescently labeled probe can be directly bound to each other via functional groups. Alternatively, linkers and first and second binding group substances may be used for indirect linkage.

### (Indirect Binding of Secondary Antibody to Fluorescently Labeled Probe)

As a method for indirectly linking the secondary antibody to the fluorescently labeled probe, for example, the secondary antibody and the fluorescently labeled probe are bound to respective linkers. Then, the secondary antibody and the fluorescently labeled probe are linked to each other by a specific binding reaction between the first binding group substance, which is attached to an end portion of one of the respective linkers, and the second binding group substance, which is attached to an end portion of the other of the respective linkers.

Examples of the first and second binding group substances include combinations of biomolecules such as biotin and avidin. Other examples of the first and second binding group substances include combinations of other known substances other than biotin and avidin (e.g., hapten and anti-hapten antibodies). Note that the first and second binding group substances do not include a substance that reacts with the first and second antibodies or antigens.

As the linkers, a known linker can be used.

The secondary antibody is modified with the first binding group substance via the linker. The fluorescently labeled probe is modified with the second binding group substance via the linker. Then, by binding the first and second binding group substances to each other, the fluorescently labeled probe is bound to the secondary antibody, and the secondary antibody is fluorescently labeled.

Note that the binding between the first and second binding group substances can be performed at a desired timing before or after the step of binding the secondary antibody to the primary antibody.

### (Direct Binding of Secondary Antibody to Fluorescently Labeled Probe)

A single linker may be interposed between the secondary antibody and the fluorescently labeled probe to link the two together, instead of binding the first and second binding group substances to each other. In this case, the fluorescently labeled probe is preferably bound to the secondary antibody before the primary antibody is bound to the antigen.

As the linker, a known linker can be used.

### (Length and Material of Linker)

In the complex of the secondary antibody and the fluorescently labeled probe bound via the linker(s), the length of the connecting portion derived from the linker(s) is preferably within a range of 15 to 1000 angstroms. The length is more preferably within a range of 30 to 65 angstroms. The linker is not particularly limited, and examples thereof include hydrophilic polymers such as polyethylene glycol, polypropylene glycol, ficoll, and polyvinyl alcohol.

### (3.1.4) Fluorescently Labeled Probe

The fluorescently labeled probe is not particularly limited as long as the fluorescently labeled probe contains a fluorescent dye and can bind to the primary antibody or the secondary antibody, and a known probe can be used. In addition, it is preferable that the fluorescently labeled probe can emit fluorescence having a sufficient intensity to represent each antigen molecule as a bright spot.

Note that in the present invention, the fluorescent dye itself is also included in the fluorescently labeled probe.

In the present invention, the “fluorescent dye” is irradiated with electromagnetic waves (X-rays, ultraviolet rays, or visible light) having a predetermined wavelength, and electrons are excited by absorbing the energy of the electromagnetic waves. Then, when the electrons return to the ground state from the excited state, the excess energy is emitted as electromagnetic waves. That is, the fluorescent dye is a substance (dye) that emits “fluorescence”. Further, in the present invention, “fluorescence” has a broad meaning. Specifically, the term “fluorescence” as used in the present application includes phosphorescence, which has a long emission lifetime and continues to emit light even after the irradiation of electromagnetic waves (excitation light) for excitation is stopped, and fluorescence in the narrow sense, which has a short emission lifetime.

The fluorescently labeled probe usable in the present invention includes, for example, particles mainly composed of an organic material or an inorganic material as a matrix, and a plurality of fluorescent dyes is encapsulated in the matrix or adsorbed onto the surface of the matrix. The fluorescently labeled probe is nanosized particles. It is preferable that the matrix and the fluorescent dye include substituent groups or moieties having charges opposite to each other, enabling an electrostatic interaction to act between the matrix and the fluorescent dye.

Examples of the organic material constituting the matrix of the fluorescently labeled probe include: resins generally classified as thermosetting resins such as melamine resin, urea resin, aniline resin, guanamine resin, phenolic resin, xylene resin, and furan resin; resins generally classified as thermoplastic resins such as styrene resin, acrylic resin, acrylonitrile resin, AS resin (acrylonitrile-styrene copolymer), ASA resin (acrylonitrile-styrene-methyl acrylate copolymer); other resins such as polylactic acid; and polysaccharides. Examples of the inorganic material include silica and glass.

### (3.1.4.1) High-brightness Fluorescent Nanoparticles

High-brightness fluorescent nanoparticles (phosphor integrated dots: PID) provide information on proteins with higher accuracy due to higher brightness of an fluorescent image when observed under a microscope.

The high-brightness fluorescent nanoparticles are a generic term for a substance having a structure in which a plurality of fluorescent dye molecules is immobilized on a resin particle by a chemical or physical action.

The particle diameter of the high-brightness fluorescent nanoparticle is not particularly limited, but for example, is preferably in a range of 10 to 500 nm, and preferably in a range of 50 to 200 nm. The coefficient of variation indicating the variation in particle diameter is not particularly limited, but is, for example, preferably 20% or less, and more preferably in a range of 5 to 15%.

For the particle diameter of the high-brightness fluorescent nanoparticle, first, an electron micrograph is taken using a scanning electron microscope (SEM). Then, the particle diameter of the high-brightness fluorescent nanoparticle can be measured by measuring the cross-sectional area of the high-intensity fluorescent nanoparticle and converting the measured value into the diameter of a circle with the same area (area circle equivalent diameter). The average particle diameter and the coefficient of variation of the high-brightness fluorescent nanoparticles are obtained by measuring the particle diameters of a sufficient number of, for example, about 1000 high-brightness fluorescent nanoparticles, as described above. Thereafter, the average particle diameter is calculated as the arithmetic mean, and the coefficient of variation is calculated by the following formula.

coefficient of variation=100×standard deviation of particle diameters/average particle diameter (%) (Formula)

### (3.1.4.1.1) Fluorescent Dye

The fluorescent dye used in the high-brightness fluorescent nanoparticles is not particularly limited.

Examples of the fluorescent dye include rhodamine-based dye molecules, squarylium-based dye molecules, cyanine-based dye molecules, aromatic hydrocarbon-based dye molecules, oxazine-based dye molecules, carbopyronine-based dye molecules, and pyromesene-based dye molecules.

Other examples include Alexa Fluor (registered trademark, manufactured by Invitrogen)-based dye molecules, BODIPY (registered trademark, manufactured by Invitrogen)-based dye molecules, Cy (registered trademark, manufactured by GE HealthCare)-based dye molecules, DY-based dye molecules (registered trademark, manufactured by Dyomics GmbH), HiLyte (registered trademark, manufactured by AnaSpec)-based dye molecules, DyLight (registered trademark, manufactured by Thermo Scientific)-based dye molecules, ATTO (registered trademark, manufactured by ATTO-TEC GmbH)-based dye molecules, and MFP (registered trademark, manufactured by MoBiTec GmbH)-based dye molecules.

### (3.1.4.1.2) Resin

The resin used for the high-brightness fluorescent nanoparticles may be a thermosetting resin or a thermoplastic resin. Among these, the resin preferably contains a melamine resin. With the melamine resin, the fluorescent dye can be immobilized inside the dense cross-linked structure, and thus the fluorescent dye can be made less likely to elute in a step using an organic solvent.

Examples of a monomer of the thermosetting resin include melamine, urea, guanamines (benzoguanamine, acetoguanamine, and the like), phenols (phenol, cresol, xylenol, and the like), xylene, derivatives thereof, and the like. These may be used alone or in combination of two or more. Furthermore, one or more comonomers other than the above-described compounds may be used in combination.

Specific examples of the thermosetting resin include a melamine-formaldehyde resin, a urea-formaldehyde resin, a benzoguanamine-formaldehyde resin, a phenol-formaldehyde resin, and a metaxylene-formaldehyde resin.

As the raw material of the thermosetting resin, a prepolymer obtained by reacting a monomer, formaldehyde, another crosslinking agent, and the like in advance may be used. For example, in the synthesis of the melamine-formaldehyde resin, generally, melamine and formaldehyde are condensed under alkaline conditions to obtain methylol melamine. Then, the methylol melamine is used as a prepolymer. The methylol melamine may be further alkyl etherified. For example, by methylation, the stability of the resin in water can be improved. In addition, by butylation, the solubility of the resin in an organic solvent can be improved.

The thermosetting resin can be synthesized according to a known method. For example, the melamine-formaldehyde resin can be synthesized by polycondensation of the pre-obtained methylol melamine by heating, if necessary, after adding a reaction accelerator such as an acid.

Examples of a monomer of the thermoplastic resin include monofunctional monomers such as styrene, (meth)acrylic acid and alkyl esters thereof, acrylonitrile, and derivatives thereof. Note that in the above examples, the thermoplastic resin has one group (vinyl group) involved in the polymerization reaction. These may be used alone or in combination of two or more. Furthermore, one or more comonomers other than the above-described compounds may be used in combination.

Specific examples of the thermosetting resin include polystyrene, styrenic resin composed of styrene and other monomers, polymethyl methacrylate, acrylic resin composed of (meth)acrylic acid and alkyl esters thereof and other monomers, polyacrylonitrile, AS resin (acrylonitrile-styrene copolymer), ASA resin (acrylonitrile-styrene-methyl acrylate copolymer), acrylonitrile resins composed of acrylonitrile and other monomers.

The thermoplastic resin may include, for example, a constituent unit, i.e., a crosslinking site formed from a polyfunctional monomer such as divinylbenzene. Note that in the above examples, the thermoplastic resin has two groups (vinyl groups) involved in the polymerization reaction. Such a thermoplastic resin includes, for example, a crosslinked product of polymethyl methacrylate.

The thermoplastic resin may include a constituent unit having a functional group for surface-modifying the obtained high-brightness fluorescent nanoparticles. For example, by using a monomer having an epoxy group, such as glycidyl methacrylate, as a raw material, it is possible to produce high-brightness fluorescent nanoparticles in which the epoxy group is oriented on the surface. This epoxy group can be converted to an amino group by reacting the epoxy group with excess aqueous ammonia. Various biomolecules can be introduced into the amino group thus formed according to a known method. Note that a molecule serving as a linker may be interposed, if necessary.

### (3.1.4.1.3) Method for Producing High-brightness Fluorescent Nanoparticles

The high-brightness fluorescent nanoparticles can be produced according to a known polymerization step (i) for various resins using a fluorescent dye that satisfies specific conditions. Furthermore, the high-brightness fluorescent nanoparticles obtained by such a method are preferably linked to a biologically-relevant binding substance by a modification step (ii).

### (A) Thermosetting Resin: Encapsulating Type

Encapsulated high-brightness fluorescent nanoparticles using a thermosetting resin can be produced basically according to an emulsion polymerization method. However, the encapsulated high-brightness fluorescent nanoparticles are preferably produced by the following polymerization step using a surfactant and a polymerization reaction accelerator. Note that in the encapsulated high-brightness fluorescent nanoparticles, it is preferable that the majority of the fluorescent dye is immobilized while being substantially encapsulated in the resin particles. However, a portion of the fluorescent dye may be immobilized while being bound to or attached to the surface of the resin particles.

In the encapsulated state of the fluorescent dye, the chemical or physical action by which the fluorescent dye is immobilized on the resin particles is not limited. If necessary, the fluorescent dye may be covalently bonded to the resin raw material in advance prior to the polymerization step. In addition, a derivatization step may be provided in order to introduce an actively charged substituent group into the resin raw material.

### (i) Polymerization Step

In the polymerization step, a reaction mixture containing a fluorescent dye, a resin raw material (a monomer, an oligomer, or a prepolymer), a surfactant, a polymerization reaction accelerator, and the like is heated to promote a polymerization reaction of the resin. Then, the resin particles that encapsulate the fluorescent dye are produced.

The order of addition of the components to be included in the reaction mixture is not particularly limited. For example, the surfactant may be added to an aqueous solution of the fluorescent dye. Subsequently, the resin raw material may be added to the reaction liquid, and finally, the polymerization reaction accelerator may be added to the reaction liquid. For another example, the resin raw material may be added to an aqueous solution of the surfactant. Subsequently, the polymerization reaction accelerator may be added to the reaction liquid, and, while a synthesis reaction of the resin particles proceeds, an aqueous solution of the fluorescent dye may be added. The concentration of the aqueous solution of the fluorescent dye used in the polymerization step is not particularly limited but is preferably in a range of 250 to 450 μM, for example.

The conditions (temperature, time, and the like) of the polymerization reaction can be appropriately set in consideration of the type of the resin, the composition of the raw material mixture, and the like. In the synthesis of the thermosetting resin such as a melamine resin, the reaction temperature is preferably within a range of 70 to 200° C., and the reaction time is preferably within a range of 20 to 120 minutes. The reaction temperature is preferably a temperature (within the heat-resistant temperature range) at which the performance of the fluorescent dye does not deteriorate. The heating may be performed in a plurality of stages. For example, after the reaction is performed at a relatively low temperature for a certain period of time, the temperature may be increased, and the reaction may be performed at a relatively high temperature for a certain period of time.

After the completion of the polymerization reaction, impurities such as excess resin raw material, fluorescent dye, and surfactant are removed from the reaction liquid, and the produced high-brightness fluorescent nanoparticles are collected and purified. For example, the reaction liquid is centrifuged to remove the supernatant containing impurities, then ultrapure water is added, and the mixture is irradiated with ultrasonic waves to be redispersed and washed. It is preferable that these procedures are repeatedly performed a plurality of times until no light absorption or fluorescence derived from the resin or the fluorescent dye is observed in the supernatant.

### (Surfactant)

As the surfactant, a known emulsifier for emulsion polymerization can be used. The surfactant includes an anionic surfactant, a nonionic surfactant, and a cationic surfactant. When a thermosetting resin having a positively charged substituent group or moiety, that is, a cationic thermosetting resin is synthesized, an anionic or nonionic surfactant is preferably used. On the other hand, when a thermosetting resin having a negatively charged substituent group or moiety, that is, an anionic thermosetting resin is synthesized, a cationic or nonionic surfactant is preferably used.

Examples of the anionic surfactant include sodium dodecylbenzene sulfonate “NEOPELEX® series” (manufactured by Kao Corp.). Examples of the nonionic surfactant include polyoxyethylene alkyl ether compounds “EMULGEN® series” (manufactured by Kao Corp.), polyvinyl pyrrolidone (PVP), and polyvinyl alcohol (PVA). Examples of the cationic surfactant include dodecyl trimethyl ammonium bromide.

By adjusting the amount of the surfactant added, the particle diameter of the resin particle can be adjusted. High-brightness fluorescent nanoparticles having a small coefficient of variation of the particle diameter, that is, having a uniform particle size can also be produced. The amount of the surfactant added is, for example, preferably in a range of 10 to 60% by mass relative to the total mass of the resin raw material. In addition, the amount is preferably in a range of 0.1 to 3.0% by mass relative to the total mass of the raw material mixture. When the amount of the surfactant added is increased, the particle diameter of the resin particles tends to be decreased. When the amount of the surfactant added is decreased, the particle diameter of the resin particles tends to be increased.

### (Polymerization Reaction Accelerator)

The polymerization reaction accelerator accelerates a polycondensation reaction of the thermosetting resin such as a melamine resin. In addition, protons (H⁺) are imparted to functional groups, such as amino groups, contained in the resin or the fluorescent dye to charge the functional groups, thereby facilitating electrostatic interactions. The reaction of the thermosetting resin progresses by heating only. However, when a polymerization reaction accelerator is added, the reaction progresses at a lower temperature. Therefore, the progress of the polymerization reaction can be controlled.

Examples of the polymerization reaction accelerator include acids such as formic acid, acetic acid, sulfuric acid, para-toluenesulfonic acid, and dodecylbenzenesulfonic acid. When the fluorescent dye is a compound having a carboxy group or a sulfo group, the fluorescent dye can also donate a proton in the same manner as the above-mentioned acids.

### (ii) Modification Step

By performing a modification step, a biologically-relevant binding substance or the like can be linked to the surface of the high-brightness fluorescent nanoparticles.

As a method for linking a biologically-relevant binding substance to the surface of the high-brightness fluorescent nanoparticles, a known method for linking a fluorescently labeled substance to a biologically-relevant binding substance can be used.

For example, a reaction occurring between reactive functional groups such as a carboxy group, an amino group, an aldehyde group, a thiol group, and a maleimide group is used. That is, one reactive functional group on the surface of the fluorescently labeled substance is bonded to the other reactive functional group in the molecule of the biologically-relevant binding substance. When these functional groups cannot be directly bonded to each other, the functional groups can be bonded via a “linker molecule” having a predetermined functional group at each of both ends of the molecule. Such a reaction can be performed by adding necessary reagents and allowing a predetermined time to pass.

As a specific example, a silane coupling agent, for example, aminopropyltrimethoxysilane is reacted with high-brightness fluorescent nanoparticles having hydroxy groups on the surface thereof to introduce amino groups. On the other hand, a thiol group-introducing reagent, for example, N-succimidyl S-acetyl thioacetic acid is reacted with streptavidin to introduce thiol groups. Finally, a PEG (polyethylene glycol)-based linker molecule having maleimide groups at both ends is reacted to link the high-brightness fluorescent nanoparticles to the streptavidin. The maleimide groups are reactive with both the amino groups and the thiol groups.

In addition, when an acrylic resin is synthesized using glycidyl methacrylate as a raw material monomer, an epoxy group derived from the monomer is present on the surface of the high-brightness fluorescent nanoparticles. Aqueous ammonia is added to the high-brightness fluorescent nanoparticles to convert the epoxy group into an amino group. The amino group can also be linked to a desired biologically-relevant binding substance or the like.

### (B) Thermosetting Resin: Adsorbing Type

A method for producing adsorbed high-brightness fluorescent nanoparticles using a thermosetting resin is as follows.

First, thermosetting resin particles without the fluorescent dye are prepared by the same procedure as in the polymerization step (1) for the encapsulated high-brightness fluorescent nanoparticles described above, except that fluorescent dye is not blended as a raw material. Subsequently, the obtained dispersion liquid of the resin particles and an aqueous solution of the fluorescent dye are mixed to cause the fluorescent dye to be adsorbed onto the surface of the resin particles. In this case, it is preferable that the thermosetting resin of the resin particles and the fluorescent dye have substituent groups or moieties having charges opposite each other and are adsorbed to each other by electrostatic interaction.

### (C) Thermoplastic Resin: Encapsulating Type

The thermoplastic resin can be synthesized according to a known method such as radical polymerization or ion polymerization (anion polymerization or the like). Encapsulated high-brightness fluorescent nanoparticles using a thermoplastic resin can also be produced according to those methods. For example, the encapsulated high-brightness fluorescent nanoparticles are preferably produced by a polymerization step according to a soap-free emulsion polymerization method.

In the polymerization step (1) in this case, a reaction mixture containing a fluorescent dye, a resin raw material, a polymerization initiator, and the like is heated to promote a polymerization reaction of the resin, thereby producing resin particles encapsulating the fluorescent dye. The polymerization initiator and the conditions (temperature, time, and the like) of the polymerization reaction can be appropriately set in consideration of the type of the resin, and the like. In the synthesis of the thermoplastic resin, the reaction temperature is preferably within a range of 20 to 150° C., and the reaction time is preferably within a range of 10 to 240 minutes.

Note that as the polymerization initiator, for example, benzoyl peroxide, azobisisobutyronitrile, or the like can be used.

### (D) Thermoplastic Resin: Adsorbing Type

A method for producing adsorbed high-brightness fluorescent nanoparticles using a thermoplastic resin is as follows.

First, thermoplastic resin particles without the fluorescent dye are prepared by the same procedure as in the polymerization step (1) for the encapsulated high-brightness fluorescent nanoparticles described above, except that fluorescent dye is not blended as a raw material. Subsequently, the obtained dispersion liquid of the resin particles and an aqueous solution of the fluorescent dye are mixed to cause the fluorescent dye to be adsorbed onto the surface of the resin particles. In this case, it is preferable that the thermoplastic resin of the resin particles and the fluorescent dye have substituent groups or moieties having charges opposite to each other and are adsorbed to each other by electrostatic interaction.

### (3.1.5) Analysis Method

An analysis method for obtaining information on the expression of the protein using the high-brightness fluorescent nanoparticles will be described. However, the analysis method is not limited thereto, and a known method can be used as the analysis method. Hereinafter, “incubate cells” means to allow a reaction to proceed that acts on the cells.

### (Fixation Process)

A sample of the cultured cells or microorganisms of the first sample is placed in each 24-well cell culture plate. Next, a tissue fixative “FB002” (manufactured by Thermo Fisher Scientific, 4% formaldehyde) is added to the sample of the cultured cells or microorganisms. The cells are incubated for 15 minutes at room temperature (25° C.). Afterwards, the cells are washed three times with phosphate-buffered saline (PBS).

### (Permeabilization Process)

Next, 0.5% Triton X-100 included in a permeabilization kit “R37602” (manufactured by Thermo Fisher Scientific) is added to the cells. The cells are incubated for 20 minutes at room temperature (25° C.). Afterwards, the cells are washed three times with phosphate-buffered saline (PBS). As a blocking treatment, 3% bovine serum albumin BSA is added, and the cells are incubated for 30 minutes at room temperature (25° C.).

### (Staining Process)

An anti-HER3 antibody “Anti-ErbB3, Intracellular, Mouse-Mono (5A12)” (manufactured by NTA) is added to the cells as the primary antibody and allowed to react overnight at 4° C. Thereafter, the cells are washed with phosphate-buffered saline (PBS).

Next, an anti-mouse IgG antibody is biotinylated using an antibody-protein labeling kit “Biotin Labeling Kit-NH2” (manufactured by Dojindo Laboratories). Then, the biotinylated anti-mouse IgG antibody is added as the secondary antibody to the cells and allowed to react.

Thereafter, the cells are fluorescently labeled with two types of fluorescent probes, that is, high-brightness fluorescent nanoparticles and “Streptavidin, Alexa Fluor 488 Conjugate” (manufactured by Thermo Fisher Scientific). Afterwards, the cells are washed three times with phosphate-buffered saline (PBS). As a nuclear staining dye, “Hoechst 33342 (Invitrogen)” (manufactured by Thermo Fisher Scientific) diluted to 10 μg/mL with phosphate-buffered saline (PBS) is added to the cells. The cells are incubated for 5 minutes at room temperature (25° C.). Afterwards, the cells are washed three times with phosphate-buffered saline (PBS). After all the staining steps are completed, the cells are encapsulated using an anti-fading encapsulant “Prolong Gold (Invitrogen)” (manufactured by Thermo Fisher Scientific) to obtain the second sample.

### (Measurement of the Number of Fluorescent Bright Spots and Brightness)

An example of a measurement method will be described.

The second sample above is photographed using, for example, a point-scanning confocal microscope “AIR+” (manufactured by Nikon Corporation). The objective lens used is 4×(NA=0.45), and the pinhole diameter is set to 31.9 μm. For example, high-definition image analysis software “Imaris (ver. 9.1.1)” (manufactured by Bitplane) is used to quantify the image photographed above. From the photographed image, the number of fluorescent bright spots and the brightness due to the high-brightness fluorescent nanoparticles are measured using, for example, a scanning electron microscope (SEM) “S4500” (manufactured by Hitachi, Ltd.). Then, the number of high-brightness fluorescent nanoparticles per cell is calculated using separately prepared calibration curves to obtain fluorescent staining measurement data.

The calibration curves can be prepared by the following method.

A dilute solution of high-brightness fluorescent nanoparticles is dropped onto a glass slide and allowed to be dried. Then, imaging is performed under the same conditions as in the case of the cells, and image processing is performed. As in the case of the cells, the same field of view as in the case of the cells is observed using SEM. The number of fluorescent bright spots and the brightness due to the high-brightness fluorescent nanoparticles are measured, and the calibration curves of the number of fluorescent bright spots and the brightness are prepared.

### (4) Step of Acquiring, from the Third Sample or the Fourth Sample, the Second Information on at Least One of Metabolites Produced by the Cells or the Microorganisms as a Result of Performing the Function, or Compositions Constituting the Cells or the Microorganisms

As a method for acquiring the second information on the metabolites of the cells or the like or the constituent compositions, a known method can be used. From the viewpoint of high accuracy, it is preferable to use a probe labeled with a luminescent substance in the present invention. Examples of the probe labeled with a luminescent substance include the following oligonucleotide probe.

### (4.1) Oligonucleotide Probe

As the probe labeled with a luminescent substance, an oligonucleotide probe described below is preferably used. By using the oligonucleotide probe, exhaustive information can be obtained without limiting the metabolite(s) or constituent composition(s) of interest. Hereinafter, the “metabolite(s) or constituent composition(s)” is also referred to as “metabolite(s) or the like”.

### (4.1.1) Composition of Oligonucleotide Probe

The “oligonucleotide probe” has a main chain that includes one or more structural units having respective sugar structures derived from pentose or hexose and respective phosphate ester bonds bonded to the respective sugar structures. The “oligonucleotide probe” includes one or more chromophores or luminophores bound to the sugar structures.

The oligonucleotide probe emits, in response to a single excitation light, two or more types of light such as fluorescence, phosphorescence, excimer emission, exciplex emission, thermally activated delayed fluorescence, excited-state intramolecular proton emission, triplet-triplet annihilation emission, twisted intramolecular charge transfer emission, and aggregated organic emission.

The oligonucleotide probe can be used for analysis of, for example, the composition of a specific target substance. In the present invention, the “specific target substance” corresponds to all the metabolites or all the constituent compositions. Specifically, when the oligonucleotide probe is allowed to interact with the metabolites or the like, the structure or electronic state of the chromophore(s) or luminophore(s) in the oligonucleotide probe changes. Then, a complex luminescence behavior different from a luminescence behavior of the oligonucleotide probe alone is obtained.

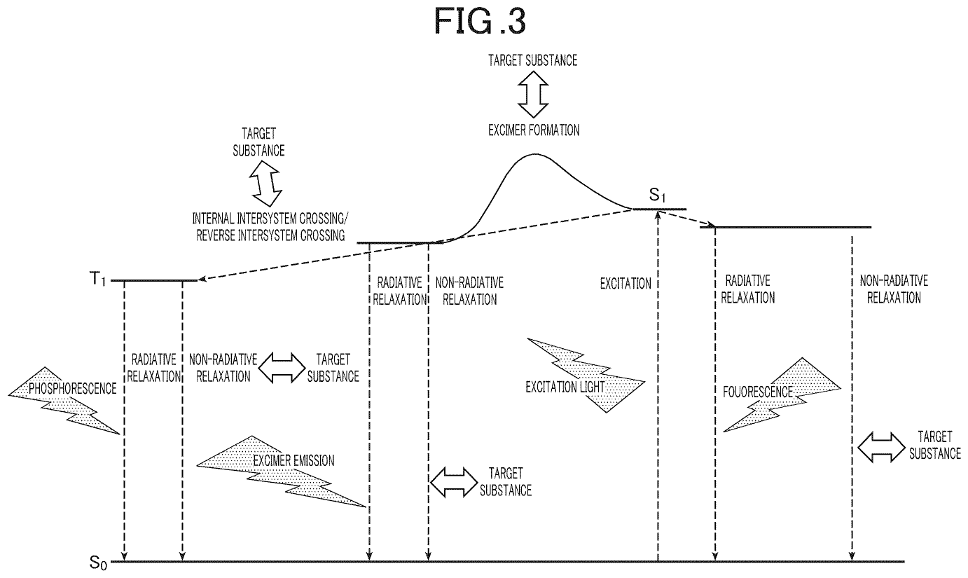

For example, as illustrated in FIG. 3, an oligonucleotide probe that emits three different types of light, that is, fluorescence, phosphorescence, and excimer emission in response to a single excitation light is allowed to interact with the target substance (metabolites or constituent compositions). Then, due to the interaction between the metabolites or the like and the oligonucleotide probe, the process in which each of the fluorescence, phosphorescence, and light emission is produced is changed, and the wavelength and lifetime of each light are changed. As a result, a large amount of complex data obtained from the combination of these types of light can be obtained according to the compositions of all the metabolites or the like. From the obtained data, it is possible to grasp the type, content and the like of the contained metabolites or constituent compositions in great detail.

It is preferable that 50% or more of the sugar structures to which the chromophores or the luminophores are bound are β-forms. In general, DNA has a structure in which bases are bonded to the main chain (deoxyribose) including phosphate ester bonds and deoxyribose-derived structures. In natural DNA, all of the deoxyribose in the main chain are in the β-form.

Therefore, when 50% or more of the sugar structures to which the chromophores or the luminophores are bound are β-forms, the similarity in structure to substances existing in nature (metabolites or the like), such as DNA and RNA, is high. Such an oligonucleotide probe is less likely to cause steric hindrance when mixed with the metabolites or the like and can penetrate into the metabolites or the like or conform to the shape of the metabolites or the like. Therefore, the metabolites or the like can be analyzed in more detail.

In the oligonucleotide probe, it is more preferable that 80% or more of the sugar structures to which the chromophores or the luminophores are bound are β-forms, and it is still more preferable that all of the sugar structures are β-forms. Whether the sugar structures to which the chromophores or the luminophores are bound are β-forms or α-forms can be confirmed by NMR analysis, X-ray crystal structure analysis, or the like. Hereinafter, a specific structure of the oligonucleotide probe will be described.

It is sufficient that the main chain of the oligonucleotide probe includes one or more structural units having respective sugar structures derived from pentose or hexose and respective phosphate ester bonds bonded to the respective sugar structures. The main chain may include only one of the above-described structural units or may include a plurality of the above-described structural units. That is, the main chain may have a structure including one sugar structure and one phosphate ester bond bonded to the sugar structure or may have a structure alternately including the sugar structure and the phosphate ester bond. Usually, both ends of the main chain of the oligonucleotide probe have the sugar structures, and thus the number of sugar structures is one more than the number of phosphate ester bonds. When the main chain includes a plurality of structural units, the plurality of structural units may be identical to or different from each other.

Furthermore, the number of the above-described structural units contained in the main chain of the oligonucleotide probe is appropriately selected according to the metabolites or the like. The number of the structural units is preferably 2 or more and 6 or less. As the amount of the structural units increases, the oligonucleotide probe becomes more likely to specifically act on the metabolites or the like. However, in the present embodiment, it is preferable to obtain a large amount of data by allowing the oligonucleotide probe to interact with various positions of the metabolites or the like. Therefore, it is preferable that the oligonucleotide probe and the metabolites or the like have moderate, that is, not excessive, specificity, and that the number of the structural units is preferably 6 or less.

The main chain of the oligonucleotide probe may partially include a structure other than the structural unit having the sugar structure derived from pentose or hexose and the phosphate ester bond, as long as that the object and the effects of the present embodiment are not impaired. The structures at both ends of the main chain are not particularly limited, and examples thereof include an OH group and an alkoxy group.

Examples of the pentose include ribose, deoxyribose, and xylose. On the other hand, specific examples of the hexose include allose, glucose, and mannose. In particular, when the sugar structure is derived from ribose or deoxyribose, the main chain of the oligonucleotide probe has the same structure as the main chain of DNA or RNA. This allows the oligonucleotide probe to interact with DNA and RNA more easily.

When the structural unit includes a structure derived from ribose or deoxyribose, the phosphate ester bond is preferably bonded to the carbon at the third position and the carbon at the fifth position of the ribose or deoxyribose. In addition, the chromophore or luminophore described below is preferably bonded to the first position of the ribose or deoxyribose. That is, the oligonucleotide probe according to the present embodiment preferably has a structure represented by the following General Formula (1a) or (1b).

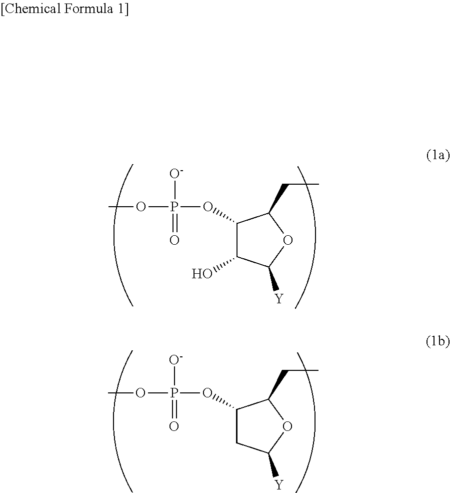

In the General Formulas (1a) and (1b), Y represents the chromophore or luminophore, which will be described below.

On the other hand, the chromophore or luminophore of the oligonucleotide probe independently emits a predetermined type of light in response to a single excitation light. It is sufficient that the structure emits a predetermined type of light by the action of a plurality of chromophores or luminophores. The chromophores or luminophores are preferably bound to the sugar structures of the main chain so that the sugar structures are β-forms.

Note that in the present specification, the term “chromophore” refers to a structure that absorbs light having a wavelength of 300 nm or more. The term “luminophore” refers to a structure that absorbs light having a wavelength of 300 nm or more to emit light.

The number of chromophores or luminophores included in the oligonucleotide probe may be only one, as long as the oligonucleotide probe can emit a plurality of types of light. However, from the viewpoint that the oligonucleotide probe is likely to emit a plurality of types of light, the number is preferably two or more, and more preferably three or more and six or less. When the oligonucleotide probe has a plurality of chromophores or luminophores, the number of types of the plurality of chromophores or luminophores may be only one or two or more.

In an oligonucleotide probe, usually one chromophore or luminophore is bound to one sugar structure in the main chain. Therefore, when the oligonucleotide probe has two or more chromophores or luminophores, it is preferable that the main chain also has two or more sugar structures. That is, it is preferable that the number of chromophores or luminophores in the oligonucleotide probe is equal to or less than the number of sugar structures in the main chain.

Note that when the number of chromophores or luminophores in the oligonucleotide probe is smaller than the number of sugar structures in the main chain, the chromophores or luminophores are not bound to some of the sugar structures. The sugar structure to which the chromophore or luminophore is not bound may not be bound to another atomic group or the like. However, a natural base may be bound to the sugar structure to which the chromophore or the luminophore is not bound, as long as the object and effects of the present embodiment are not impaired. Examples of the natural base include adenine, guanine, cytosine, thymine, and uracil.

The total number of natural bases bound to the sugar structures is preferably 50% or less, and more preferably 25% or less, relative to the total number of sugar structures included in the main chain. When the number of natural bases is 50% or less, association between the oligonucleotide probes is suppressed, and interaction between the metabolites and the oligonucleotide probe tends to be dominant. In addition, it is preferable that the natural bases and the non-natural bases are bound so that the sugar structures are β-forms.

Examples of chromophores or luminophores that emit fluorescence include structures derived from fluorescein, rhodamine, boron dipyrromethene, and the like. Examples of chromophores or luminophores that emit phosphorescence include structures derived from iridium complexes, platinum complexes, and the like. Examples of chromophores or luminophores that emit excimer emission include structures derived from pyrene, anthracene, perylene, and the like. Examples of chromophores or luminophores that emit exciplex emission include structures derived from pyrene-dimethylaniline and the like.

Examples of chromophores or luminophores that emit thermally activated delayed fluorescence include structures derived from 4CzIPN, DABNA, and the like. Examples of chromophores or luminophores that emit excited-state intramolecular proton emission include structures derived from hydroxyphenylbenzoxazole and the like. Examples of chromophores or luminophores that emit triplet-triplet annihilation emission include structures derived from 9,10-diphenylanthracene, rubrene, and the like. Examples of chromophores or luminophores that emit twisted intramolecular charge transfer emission include structures derived from diaminoanthracene, diaminonaphthalene, and the like. Examples of chromophores or luminophores that emit aggregated organic emission include structures derived from tetraphenylethene, hexaphenylsilole, and the like.

Among them, the oligonucleotide probe preferably has, as the chromophore(s) or the luminophore(s), at least one of a structure that emits fluorescence, a structure that emits excimer emission, or a structure that emits exciplex emission. In particular, the oligonucleotide probe preferably has at least a structure that emits fluorescence. When the oligonucleotide probe emits fluorescence, there is an advantage that the oligonucleotide probe can be easily analyzed by various measuring apparatuses.

It is preferable that the oligonucleotide probe emits a plurality of types of light when irradiated with light in a wavelength range of 300 to 400 nm. When the oligonucleotide probe emits a plurality of types of light when irradiated with light in the above wavelength range, a special light source is not required, and the target object is less likely to be damaged by light when the target object is analyzed.

The molecular weight of the oligonucleotide probe is appropriately selected depending on the type of chromophore(s) or luminophore(s) contained in the oligonucleotide probe, the length of the main chain, and the like. Usually, the molecular weight is preferably within a range of 500 to 10000, and more preferably within a range of 100 to 4000. When the molecular weight of the oligonucleotide probe is 10000 or less, the specificity to the target object is moderately low, and the oligonucleotide probe can nonspecifically react with a plurality of sites of the target object.

### (4.1.2) Method for Producing Oligonucleotide Probe

The oligonucleotide probe is synthesized by first preparing monomers in which chromophores or luminophores and phosphate esters are bonded to pentoses or hexoses. Then, the monomers are polymerized in a desired sequence using a phosphoramidite method with a DNA/RNA synthesizer or the like to obtain the oligonucleotide probe. By using such a method, a plurality of types of monomers having different types of chromophores or luminophores are prepared, and the sequence of the monomers is changed to bind a desired number of monomers.

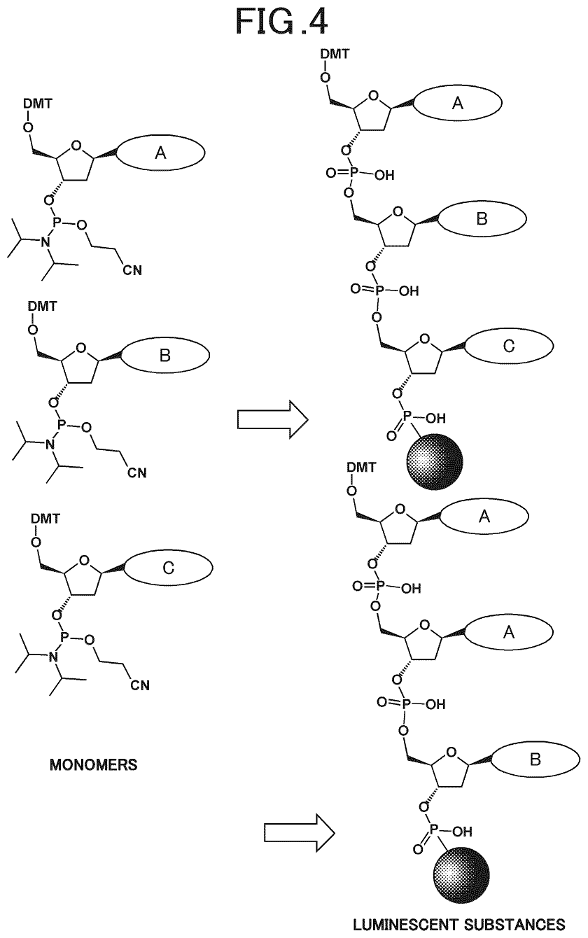

For example, as illustrated in FIG. 4, first, three types of monomers in which the chromophores or the luminophores are A, B, and C, respectively, are prepared. Then, three monomers are selected from among them, and the type of the monomers to be selected and the sequence of the monomers are changed to bind the monomers. This allows 27 different types of oligonucleotide probes to be synthesized.

That is, a wide variety of oligonucleotide probes can be synthesized from a plurality of types of monomers having different types of chromophores or luminophores. By changing the type of monomers used and the number of monomer bonds, a great variety of oligonucleotide probes can be synthesized.

Usually, in the phosphoramidite method, a portion of a monomer, for example, a hydroxy group of a sugar structure or a hydroxy group derived from phosphoric acid is supported on a particulate carrier to perform a polymerization reaction. The hydroxy group of the sugar structure is, for example, a hydroxy group bonded to the carbon at the third position of ribose or deoxyribose. In addition, the particulate carrier is also referred to as a “carrier particle” in the present specification.

Examples of the carrier particle include porous glass and polystyrene. After the synthesis of the oligonucleotide probe (polymerization of monomers), the carrier particle may be removed to obtain only the oligonucleotide probe. The oligonucleotide probe may be used or distributed while the oligonucleotide probe is supported on the carrier particle. In the present specification, this is also referred to as a “luminescent substance carrier”.

### (4.1.3) Effect of Use of Oligonucleotide Probe

The oligonucleotide probe has the main chain structure similar to that of substances found in nature, such as DNA and RNA. The oligonucleotide probe can then easily interact with various metabolites and the like. Therefore, without limiting the target metabolite, all the metabolites or the like can be targeted, that is, the metabolites or the like can be exhaustively analyzed.

By using the oligonucleotide probe, the compositions of the metabolites or the like can be grasped in detail. In addition, the oligonucleotide probe emits a plurality of types of light when irradiated with light having a specific wavelength. Therefore, it is possible to obtain a large amount of complex luminescence data according to the compositions of the metabolites or the like, and it is possible to analyze the metabolites or the like in very detail.

### (4.2) Analysis Method

An example of the analysis method for the metabolites (third sample) using the above-described oligonucleotide probe is described below. However, the analysis method using the oligonucleotide probe is not limited thereto.

In addition, by using the fourth sample instead of the third sample, the constituent compositions can be analyzed in a similar manner. Furthermore, by using a mixture of the third sample and the fourth sample instead of the third sample, the metabolites and the constituent compositions can be analyzed as a whole.

### (4.2.1) Steps in Analysis Method

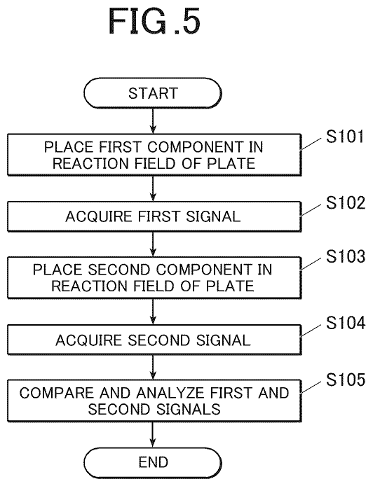

A flowchart of the example of the analysis method is illustrated in FIG. 5.

First, one of the oligonucleotide probe and the third sample (metabolites) is placed on a reaction field of a plate. This step is referred to as a “first component placement step (S101)”. The placed component is also referred to as a “first component” below. The term “reaction field” refers to a field for allowing the oligonucleotide probe and the metabolites to interact with each other.

Next, first signal information is acquired from the plate on which the first component is placed. This step is referred to as a “first signal information acquisition step (S102)”.

The other of the oligonucleotide probe and the metabolites is further placed on the reaction field of the plate from which the first signal information has been acquired. This step is referred to as a “second component placement step (S103)”. The component placed in this step is also referred to as a “second component”.

Second signal information is acquired from the plate on which the oligonucleotide probe and the metabolites are placed. This step is referred to as a “second signal acquisition step (S104)”.

Thereafter, the first signal information and the second signal information are compared and analyzed by an analysis section. This step is referred to as an “analysis step (S105)”.

The type of the metabolites to be analyzed by the analysis method according to the present embodiment is not particularly limited. For example, the metabolites may be substances whose structure is known or substances whose structure is unknown. In addition, a plurality of metabolites may be mixed. In the present invention, it is preferable to exhaustively analyze a plurality of metabolites whose structures are unknown.

Hereinafter, the analysis method according to the present embodiment will be described in detail. In the following description, a case where the oligonucleotide probe is placed in the reaction field in the first component placement step and the third sample (metabolite) is placed in the reaction field in the second component placement step will be described as an example. However, the analysis method according to the present embodiment is not limited thereto.

### (4.2.1.1) First Component Placement Step

In the first component placement step, the oligonucleotide probe is placed in a reaction field of a plate having the reaction field for allowing the metabolites and the oligonucleotide probe to interact with each other.

The plate used in this step only needs to have the reaction field, and the number of reaction fields may be one or two or more. From the viewpoint of analyzing a plurality of metabolites or analyzing a plurality of metabolites using a plurality of oligonucleotide probes, the number of reaction fields of one plate is preferably plural. When the plate has a plurality of reaction fields, the plurality of reaction fields is preferably spaced apart. The plate may have a flat plate shape or may have unevenness depending on the shape of the reaction field. The material, size, shape, and the like of the plate are appropriately selected depending on the use of analysis, the types of the oligonucleotide probe and the metabolites, and the like.

When the plate has a plurality of reaction fields, the positions of the reaction fields are preferably set at intervals so that adjacent reaction fields do not contact each other. The interval is appropriately selected depending on the size of the reaction fields, the types of the oligonucleotide probe and the metabolites, and the like.

When the first component placement step and the second component placement step are performed by a machine (e.g., an inkjet device or the like), a mark (formation of an uneven structure or marking) or the like indicating the position of each reaction field may not be formed on the plate. On the other hand, when a mark (formation of an uneven structure or marking) indicating the position of each reaction field is formed on the plate, it is easy to accurately place the metabolites and the oligonucleotide probe at a desired position (reaction field).

When each reaction field is formed in a recessed shape or when a partition wall is placed around each reaction field, the metabolites and the oligonucleotide probe placed in adjacent reaction fields are less likely to be mixed, and more accurate analysis is easily performed. Furthermore, for example, when a water-repellent treatment portion is positioned around a reaction field, the metabolites and the oligonucleotide probe in adjacent reaction fields are less likely to be mixed, and more accurate analysis is easily performed. In the present embodiment, it is preferable to use a plate in which a plurality of wells (reaction fields) is arranged in a regular pattern. In a plate having such wells, the wells are physically separated from each other by partition walls. This makes it difficult for the metabolites and the oligonucleotide probe in adjacent reaction fields to be mixed, facilitating accurate analysis.

The number of reaction fields in one plate is appropriately selected depending on the type of object to be analyzed, the type of the oligonucleotide probe, and the like. The number of reaction fields is not particularly limited, but as the number of reaction fields increases, a larger number of multidimensional data can be acquired, and more precise analysis can be performed.

The method for placing the first component, which is the oligonucleotide probe in the present embodiment, in each reaction field is not particularly limited and is appropriately selected depending on the type, physical property, and the like of the first component. Examples of the method for placing the first component include application by an inkjet device, application by a dispenser, placement of a carrier that supports the first component, and direct fixation of the first component to the reaction field. Among these, the inkjet method is particularly preferable. By using the inkjet method, the first component (oligonucleotide probe) in liquid form can be efficiently placed in a large number of regions (reaction fields) to form the reaction fields. Thus, a large amount of data can be acquired.

When the plate has a plurality of reaction fields, the same first component (oligonucleotide probe) may be placed in all of the plurality of reaction fields. A plurality of types of first components (oligonucleotide probes) may be placed in the same reaction field. First components (oligonucleotide probes) having different compositions from each other may be placed in two or more reaction fields. When different types of first components (oligonucleotide probes) are placed respectively in different reaction fields, a plurality of types of interactions between the oligonucleotide probes and the metabolites occur. Then, the metabolites can be analyzed in more detail.

### (4.2.1.2) First Signal Information Acquisition Step

In the first signal information acquisition step, the first signal information is acquired from the plate on which the first component is placed in the reaction field. The first signal information acquired in this step is not particularly limited as long as the information is useful for the analysis described below.

The oligonucleotide probe emits a plurality of types of light in response to a single excitation light. For example, the oligonucleotide probe may be irradiated with a specific excitation light (excitation light having a single wavelength), and the intensity and/or wavelength of light emitted from the oligonucleotide probe (emission information) in response to the excitation light may be acquired as the first signal information. Furthermore, a change over time in the spectral distribution or chromaticity of light emitted from the oligonucleotide probe when irradiated with a specific excitation light may be acquired as the first signal. The data acquired in the first signal information acquisition step may be only one type of data or may be two or more types.

When the intensity and/or wavelength of light emitted from the oligonucleotide probe is to be acquired, the oligonucleotide probe is irradiated with excitation light having a single wavelength. Then, the emission intensity and emission wavelength of the oligonucleotide probe may be acquired using a general spectrophotometer or the like. When the spectral distribution change of the oligonucleotide probe is to be acquired, the oligonucleotide probe is irradiated with excitation light having a single wavelength only for a short time. Then, the light emitted from the oligonucleotide probe in response may be continuously or intermittently acquired by a spectrophotometer or the like.

Furthermore, when the chromaticity change of light emitted from the oligonucleotide probe is to be acquired, the oligonucleotide probe is irradiated with excitation light having a single wavelength only for a short time. Then, the light emitted from the oligonucleotide probe in response may be acquired as an image using a CCD camera, a CMOS camera, or the like. By identifying chromaticity from the acquired image, data on the chromaticity change over time can be obtained.

### (4.2.1.3) Second Component Placement Step

In the second component placement step, the other of the oligonucleotide probe and the metabolites, which is the metabolites in the present embodiment, is placed in the reaction field from which the first signal information has been acquired. When the plate has a plurality of reaction fields, second components (metabolites) having different compositions may be placed in some or all of the reaction fields. On the other hand, a second component (metabolites) having the same composition may be placed in all the reaction fields.

Note that the method for placing the second component (metabolites) is not particularly limited and is appropriately selected depending on the type and properties of the second component. As the method for placing the second component, the same method as the method for placing the first component can be used. In the second component placement step, the second component may also be placed in a region where the first component has not been placed.

### (4.2.1.4) Second Signal Information Acquisition Step

In the second signal information acquisition step, the second signal information is acquired from the plate on which the second component has been placed. The second signal information acquired in this step is not particularly limited as long as the information is useful for the analysis in the analysis step described below. Usually, it is preferable to acquire the second signal information in the same manner as the information acquired in the first signal information acquisition step.

### (4.2.1.5) Analysis Step

In the analysis step, the first signal information acquired in the first signal information acquisition step and the second signal information acquired in the second signal information acquisition step are compared to analyze the target object. Specifically, first, data is obtained by subtracting the first signal information from the second signal information. Hereinafter, this data is also referred to as “analysis data”. Then, the compositions or the like of the metabolites are analyzed based on the size, value and the like of the analysis data. An analysis method for the analysis data in the present step is appropriately selected depending on the purpose, the type of the analysis data, and the like.

For example, the same steps as the first component placement step, the first signal information acquisition step, the second component placement step, the second signal information acquisition step, and the like are performed in advance for ideal metabolites. Then, standard data is prepared. The compositions or the like of the metabolites may be identified by comparing the standard data with the analysis data. In addition, when the metabolite is composed of a plurality of components or when a plurality of parameters is involved, standard data is prepared for when the target object is in good condition and when the target object is in bad condition. Then, the standard data may be compared with the analysis data.

When the analysis is performed, the standard data may be simply compared with the analysis data. Further, for example, the comparison results between the standard data and the analysis data may be converted into a distance matrix and analyzed by a heat map (without weighting). The distance matrix may be subjected to principal component analysis (also referred to as PCA, weighting with emphasis on anisotropy), analysis by DL (weighting with emphasis on isotropy), or the like.

On the other hand, the standard data may be a trained model generated in advance by machine learning or the like. The trained model can be created by, for example, a machine learning step described below, but the trained model to be used is not limited to a model created in the machine learning step described below. When the trained model is used, more appropriate analysis can be performed on the target object.

When the trained model is referred to, the analysis data is applied to the trained model. Then, it is possible to determine (predict), from accumulated data and the like, whether the metabolite has a desired composition, how much of a predetermined structure the metabolite contains, whether the target object is in good condition, and the like. The prediction result may be obtained as, for example, classification, regression, clustering, or anomaly detection (outlier detection).

### (4.2.1.6) Machine Learning Step

The analysis method according to the present embodiment may further include a learning step of machine learning the first signal information and the second signal information to generate a trained model.

For example, in the machine learning step, a plurality of prediction models is constructed based on the difference (analysis data) between the second signal information and the first signal information. Then, results of the plurality of prediction models are combined to create a trained model that can predict information on the metabolites (e.g., compositions).

When the structure and the amount of the contained metabolite are known in advance, a prediction model can be constructed by performing machine learning using the characteristics of the analysis data as explanatory variables and the structure and amount of the contained metabolite as target variables. As the explanatory variables, numerical values representing the characteristics of the analysis data or numerical values calculated therefrom can be used. When the first signal information or the second signal information is a spectral distribution, the intensity of light for each wavelength or the like can be used as an explanatory variable. On the other hand, the target variables can be appropriately selected according to the purpose of analysis and are not limited to the structure and the amount of the contained metabolite. The target variables may be any other variables related to the metabolites.

The machine learning performed in this step may be supervised learning or may be unsupervised learning. The term “supervised learning” refers to a learning method in which the “relationship between input and output” is learned from training data with correct answer labels. The term “unsupervised learning” refers to a learning method in which the “structure of a data set” is learned from training data without correct answer labels.

Furthermore, the machine learning may be reinforcement learning, deep learning, or deep reinforcement learning. The term “reinforcement learning” refers to a learning method in which an “optimal sequence of actions” is learned through trial and error. The term “deep learning” refers to a learning method in which, from a large amount of data, characteristics contained in the data are learned in stages more deeply (at a deeper level). The term “deep reinforcement learning” refers to a learning method in which the reinforcement learning and the deep learning are combined.

A general analysis method (algorithm) can be applied to the machine learning. For the machine learning, for example, a prediction model constructed by an analysis method selected from linear regression (multiple regression analysis, partial least squares (PLS) regression, LASSO regression, Ridge regression, principal component regression (PCR), and the like), random forest, decision tree, support vector machine (SVM), support vector regression (SVR), neural network, discriminant analysis, and the like can be applied.

### (4.2.2) Effect of Use of Analysis Method

In the analysis method using the oligonucleotide probe, the oligonucleotide probe and the target object are allowed to interact with each other to acquire the first signal information and the second signal information. Then, by analyzing the first signal information and the second signal information, various information on the metabolites contained in the target object can be obtained. By obtaining signal information on the target object as a whole in which a plurality of metabolites are contained, information on the cells or the like can be obtained without needing to specify the type, content, and the like of the metabolites.

### (5) A Step of Analyzing at Least the Function or the State of the Cell or the Microorganism Based on the Obtained Information on the Cell or the Microorganism

The function of the cells or the like can be analyzed from the information on the expression of the protein. In addition, it is possible to analyze the degree to which the cells or the like performs the function, that is, the state of the cells or the like, based on the information on the metabolites or the like.

In addition, it is preferable to apply the machine learning as described in the analysis method using the oligonucleotide probe not only to the information on the metabolites or the like but also to the information on the expression of the protein.

### 7. Analysis System

The analysis system according to the present invention is an analysis system for a cell or a microorganism and is characterized by using the above-described analysis method.

The “analysis system” according to the present invention refers to an assembly of devices, apparatuses, and the like having predetermined functions that are used as means elements necessary in each step of the analysis of the cell or the like. Specifically, the analysis system includes devices, apparatuses, and the like used for the preparation of the first to fourth samples and devices, apparatuses, and the like used for the analysis of the second to fourth samples.

Then, these as a whole perform the function of analyzing the cell or the like. The means elements may be individually disposed at different places apart from each other. Furthermore, the means elements may be collectively disposed in a certain space as a single apparatus to be a system apparatus.

### EXAMPLES

Hereinafter, the present invention will be specifically described with reference to Examples, but the present invention is not limited thereto. In Examples, “part(s)” or “%” means “part(s) by mass” or “% by mass” unless otherwise specified.

In the following Examples, operations were performed at room temperature (25° C.) unless otherwise specified.

In the present Examples, in order to verify the effects of the present invention, two types of drugs that affect cell metabolism and protein expression were added to a cell culture solution in different combinations as a model system. Next, cell states having different protein expression amounts and different metabolite compositions were set under six conditions from Type1 to Type6. Then, 10 wells were cultured under each condition, a total of 60 specimens were measured, and the results were analyzed.

### 1. Preparation of First Sample

### (1) Cell Culture

HER2 high expressing human breast cancer culture strain SKBR-3 (American Type Culture Collection: ATCC) was cultured for three days according to the ATCC recommended protocol. The culture solution used was Dulbecco Modified Eagle Medium (DMEM) supplemented with fetal bovine serum (FBS). The amount of FBS added was 10% by mass relative to the total mass of DMEM.

Thereafter, the cultured cells were collected into 24-well cell culture plates so that 5×10⁴ cells were collected per well. Then, the cells were cultured for 16 to 18 hours under conditions of a temperature of 37° C. and a carbon dioxide concentration of 5% by volume.

### (2) Addition of Drugs to Cultured Cells

An experiment was performed in which drugs were added to the cultured cells in vitro. As the drugs, “Gefitinib” (manufactured by Funakoshi Co., Ltd.) and “7-ethyl-10-hydroxycamptothecin (SN-38)” (manufactured by Tokyo Chemical Industry Co., Ltd.) were used. Each drug was added to the cultured cells so that the concentration of each drug in the culture solution was as described in Table III. The final concentration of dimethyl sulfoxide (DMSO) in each culture solution was adjusted to 0.1% by mass. The cells were cultured for 24 hours under conditions of a temperature of 37° C. and a carbon concentration of 5% by volume to obtain the first sample.

Table III shows the conditions for producing six types of cells in different states.

TABLE III

CONCENTRATION OF DRUG

| | GEFITINIB | SN-38 |
|---|---|---|
| | [μM] | [μM] |
| Type1 | 5.00 | 0.01 |
| Type2 | 1.00 | 0.00 |
| Type3 | 0.00 | 0.01 |
| Type4 | 5.00 | 0.00 |
| Type5 | 1.00 | 0.01 |
| Type6 | 0.00 | 0.00 |

### 2. Preparation of Second Sample (Sample for Acquiring Protein Information)

### (Fixation Process)

The culture solution in each of the 24-well cell culture plates was collected with an aspirator and dispensed into a separate container. A tissue fixative “FB002” (manufactured by Thermo Fisher Scientific, 4% formaldehyde) was added to the cells in each of the 24-well cell culture plates. The cells were incubated for 15 minutes at room temperature (25° C.). Thereafter, the cells were washed three times with phosphate-buffered saline (PBS).

### (Permeabilization Process)

Next, 0.5% Triton X-100 included in a permeabilization kit “R37602” (manufactured by Thermo Fisher Scientific) was added to the cells. The cells were incubated for 20 minutes at room temperature (25° C.). Thereafter, the cells were washed three times with phosphate-buffered saline (PBS). As a blocking treatment, 3% bovine serum albumin BSA was added, and the cells were incubated for 30 minutes at room temperature (25° C.).

### (Staining Process)

An anti-HER3 antibody “Anti-ErbB3, Intracellular, Mouse-Mono (5A12)” (manufactured by NTA) was added to the cells as the primary antibody and allowed to react for 16 to 18 hours at 4° C. Thereafter, the cells were washed with phosphate-buffered saline (PBS).

Next, an anti-mouse IgG antibody “Goat Anti-Mouse IgG” (manufactured by Tokyo Chemical Industry Co., Ltd.) was biotinylated using an antibody-protein labeling kit “Biotin Labeling Kit-NH2” (manufactured by Dojindo Laboratories). Then, the biotinylated anti-mouse IgG antibody was added as the secondary antibody to the cells and allowed to react for 30 minutes at room temperature.

Thereafter, the cells were fluorescently labeled with each of two types of fluorescently labeled probes, that is, high-brightness fluorescent nanoparticles described later and “Streptavidin Alexa Fluor 488 Conjugate” (manufactured by Thermo Fisher Scientific). Thereafter, the cells were washed three times with phosphate-buffered saline (PBS). As a nuclear staining dye, “Hoechst 33342 (Invitrogen)” (manufactured by Thermo Fisher Scientific) diluted to 10 μg/mL with phosphate-buffered saline (PBS) was added to the cells. The cells were incubated for 5 minutes at room temperature (25° C.). Thereafter, the cells were washed three times with phosphate-buffered saline (PBS). After all the staining steps were completed, the cells were encapsulated using an anti-fading encapsulant “Prolong Gold (Invitrogen)” (manufactured by Thermo Fisher Scientific) to obtain the second sample.

### 3. Acquisition of Protein Information

### (1) Production of High-brightness Fluorescent Nanoparticles (PID)

Encapsulated high-brightness fluorescent nanoparticles with accumulated perylenediimide, which is a fluorescent dye, were prepared using the procedure described in “Quantitative diagnostic imaging of cancer tissues by using phosphor-integrated dots with ultra-high brightness,” Scientific Reports, volume 7, Article number 7509, 2017, and surface-modified with streptazibine. As the resin, polymethyl methacrylate-based nano-crosslinked fine particles “EPOSTAR (registered trademark) MX030W” (manufactured by Nippon Shokubai Co., Ltd.) were used. An SEM image of the obtained 300 high-brightness fluorescent nanoparticles was obtained using a scanning electron microscope “S-4100” (manufactured by Hitachi, Ltd.). Then, the particle diameters were measured from the image, and the average value thereof was calculated. The average particle diameter was 132 nm. The fluorescence properties of the obtained high-brightness fluorescent nanoparticles were also measured using a fluorometer “F-7000” (manufactured by Hitachi, Ltd.). The maximum excitation wavelength was 589 nm and the maximum fluorescence wavelength was 621 nm.

### (2) Fluorescent Dye

As the probe for the fluorescent dye alone, “Streptavidin Alexa Fluor 488 Conjugate” (manufactured by Thermo Fisher Scientific) was used.

### (3) Measurement of the Number of Fluorescent Bright Spots and Brightness

The second sample was photographed using a point-scanning confocal microscope “AIR+” (manufactured by Nikon Corporation). The objective lens used was 4×(NA=0.45), and the pinhole diameter was set to 31.9 μm. High-definition image analysis software “Imaris (ver. 9.1.1)” (manufactured by Bitplane) was used to quantify the image photographed above. From the photographed image, the number of fluorescent bright spots and the brightness were measured using a scanning electron microscope (SEM) “S4500” (manufactured by Hitachi, Ltd.). Then, the number of high-brightness fluorescent nanoparticles or the fluorescent dye per cell was calculated using separately prepared calibration curves to obtain fluorescent staining measurement data. The average value of 10 wells for the number of bright spots per cell is shown in Table IV.

The calibration curves were prepared by the following method.

A dilute solution of high-brightness fluorescent nanoparticles or the fluorescent dye was dropped onto a glass slide and allowed to be dried. Then, imaging was performed under the same conditions as in the case of the cells, and image processing was performed. As in the case of the cells, the same field of view as in the case of the cells was observed using SEM. The number of fluorescent bright spots and the brightness were measured, and the calibration curves of the number of fluorescent bright spots and the brightness were prepared.

TABLE IV

| | | | AVERAGE VALUE |
|---|---|---|---|
| | CONCENTRATION OF DRUG | | OF 10 WELLS FOR |
| | GEFITINIB | SN-38 | NUMBER OF BRIGHT |
| | [μM] | [μM] | SPOTS PER CELL |
| Type1 | 5.00 | 0.01 | 451 |
| Type2 | 1.00 | 0.00 | 152 |
| Type3 | 0.00 | 0.01 | 12 |
| Type4 | 5.00 | 0.00 | 339 |
| Type5 | 1.00 | 0.01 | 132 |
| Type6 | 0.00 | 0.00 | 13 |

### 4. Preparation of Third Sample (Sample for Acquiring Information on Metabolite) and Preparation of Fourth Sample (Sample for Acquiring Information on Composition that Constitutes the Cell)

The culture solution that was collected by an aspirator and dispensed into the separate container in the preparation of the second sample above was used as the third sample.

The fourth sample was prepared by the following method.

A portion of the cells cultured in the “(1) Cell Culture” in the step of the “preparation of the first sample” was collected. Next, the collected cells were washed with phosphate-buffered saline (PBS) by centrifugation. Next, the washed cells were homogenized using an ultrasound disperser. A required amount of RIRA buffer (manufactured by Nacalai Tesque, Inc.) was added to the cells to prepare the sample (fourth sample) of the crushed cell liquid.

### 5. Acquisition of Information on Metabolites and Compositions that Constitute Cells

### (1) Preparation of Oligonucleotide Probe

In the preparation of the oligonucleotide probe, all reactions were performed under a nitrogen atmosphere in oven-dried glassware unless otherwise specified. All chemical products were purchased from Aldrich, TCI, or Kanto Chemical Co., Inc. and used as supplied without further purification.

### (1.1) Synthesis of Monomer 1

Based on the following reaction formula, a monomer 1 having a main chain containing a phosphate ester and a luminophore bound to the main chain was synthesized via intermediates 1 to 6.

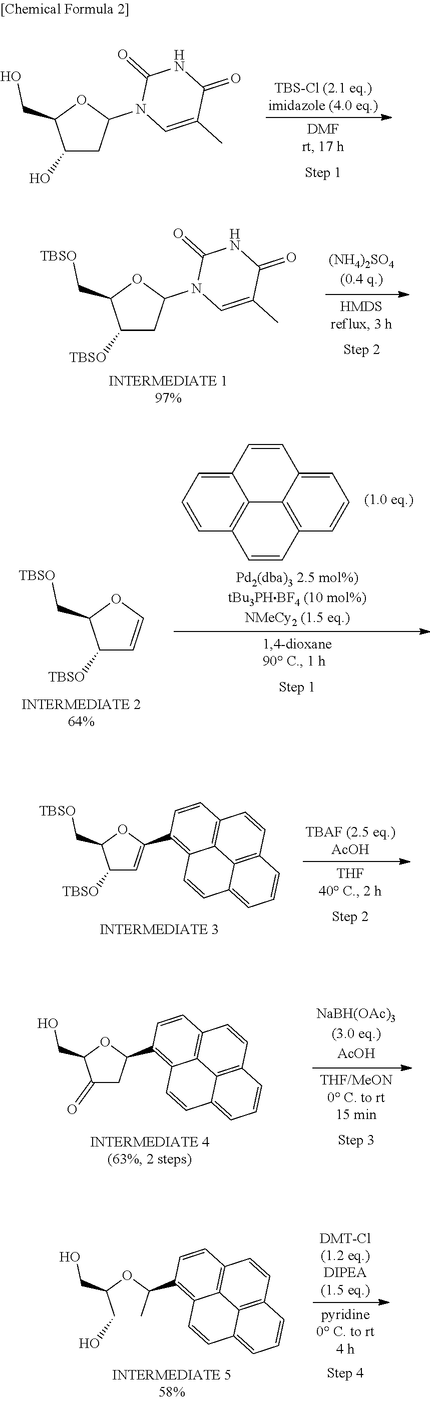

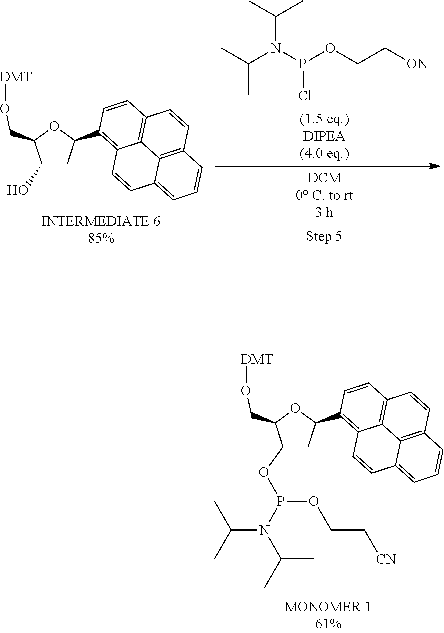

### Synthesis of Intermediate 1

Thymidine (15.0 g, 61.9 mmol) and imidazole (16.9 g, 248 mmol) were dissolved in dimethylformamide (DMF) (124 mL). To the reaction solution was added tert-butyl dimethylsilyl chloride (19.6 g, 130 mmol), and the mixture was stirred for 17 hours at room temperature. Water was added to the reaction solution, and the mixture was subjected to liquid-liquid extraction with ethyl acetate. The obtained organic phase was dried over magnesium sulfate, and the solvent was distilled off. Then, the target intermediate 1 was obtained as a colorless solid (28.3 g, 97%).

### Synthesis of Intermediate 2

The intermediate 1 (28.3 g, 60.1 mmol) and ammonium sulphate (12.7 g, 96.2 mmol) were dissolved in hexamethyldisilazane (314 mL, 1.50 mol). The reaction solution was heated to reflux for 3 hours. Thereafter, water was added to the reaction solution, and the mixture was subjected to liquid-liquid extraction with ethyl acetate. The obtained organic phase was dried over magnesium sulfate, and the solvent was distilled off. The obtained crude product was purified by silica gel column chromatography to obtain the target intermediate 2 as a brown liquid (13.2 g, 64%).

### Synthesis of Intermediate 4

A mixture of the intermediate 2 (10.1 g, 29.3 mmol), 1-bromopyrene (8.24 g, 29.3 mmol), tris(dibenzylideneacetone) dipalladium (0) (671 mg, 733 μmol), tri-tert-butylphosphonium tetrafluoroborate (850 mg, 2.93 mmol), dicyclohexylmethylamine (9.35 mL, 44.0 mmol), and 1,4-dioxane (100 mL) was heated at 90° C. for 1 hour. Water was added to the reaction solution to stop the reaction, and the mixture was subjected to liquid-liquid extraction with ethyl acetate. The obtained organic phase was dried over magnesium sulfate, and the solvent was distilled off. A crude product containing the intermediate 3 was obtained. The crude product containing the intermediate 3 was used directly in the next reaction.

To the crude product containing the intermediate 3 were added 100 mL of tetrahydrofuran (THF), 1M tetrabutylammonium fluoride THF solution (117 mL, 117 mmol), and acetic acid (6.74 mL, 117 mmol), and the mixture was stirred at 40° C. for 2 hours. Water was added to the reaction solution to stop the reaction, and the mixture was subjected to liquid-liquid extraction with ethyl acetate. The obtained organic phase was dried over magnesium sulfate, and the solvent was distilled off. The obtained crude product was purified by silica gel column chromatography to obtain the target intermediate 4 as a light brown solid (5.87 g, 63%).

### Synthesis of Intermediate 5

Sodium triacetylborate (11.8 g, 55.8 mmol) and acetic acid (7.87 mL, 138 mmol) were dissolved in 93 mL of acetonitrile. The reaction solution was cooled to 0° C. A solution of the intermediate 4 (5.87 g, 18.6 mmol) in THF (62 mL) was added dropwise to the reaction solution. After completion of the dropwise addition, the reaction solution was warmed to room temperature and stirred for 15 minutes. Water was added to the reaction solution to stop the reaction, and the mixture was subjected to liquid-liquid extraction with ethyl acetate. The obtained organic phase was dried over magnesium sulfate, and the solvent was distilled off. The obtained crude product was purified by silica gel column chromatography and reverse phase HPLC to obtain the target intermediate 5 as a colorless solid (3.44 g, 58%).

### Synthesis of Intermediate 6

A mixture of the intermediate 5 (3.44 g, 10.8 mmol), 4,4′-dimethoxytrityl chloride (4.40 g, 13.0 mmol), ethyl diisopropylamine (2.82 mL, 16.2 mmol) and dehydrated pyridine (54 mL) was stirred at room temperature for 4 hours. Methanol was added to the reaction solution to stop the reaction, and the solvent was distilled off. The obtained crude product was purified by silica gel column chromatography to obtain the target intermediate 6 as a colorless viscous solid (5.71 g, 85%).

### Synthesis of Monomer 1

To a mixture of the intermediate 6 (5.71 g, 9.20 mmol), ethyl diisopropylamine (6.42 mL, 36.8 mmol), and dehydrated dichloromethane (92 mL) was added dropwise 2-cyanoethyl diisopropyl chlorophosphoroamidite (3.08 mL, 13.8 mmol) at 0° C. The reaction solution was warmed to room temperature and stirred for 3 hours, and the solvent was distilled off. The obtained crude product was purified by silica gel column chromatography to obtain the target monomer 1 as a colorless solid (4.64 g, 61%).

### (1.2) Preparation of Monomer 2

A monomer 2 was purchased from Glen Research (Sterling, Va.) as a reagent with the structure shown below.

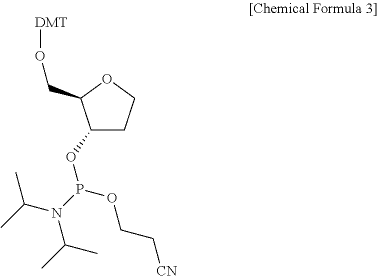

### (1.3) Synthesis of Oligonucleotide Probes 1 to 16

16 types of mixed sequence oligonucleotides (Seq1 to Seq16) of the monomer 1 and the monomer 2 were synthesized according to a conventional method, as shown in Table V below. DNA synthesis reagents were purchased from Glen Research (Sterling, Va.). All oligonucleotides were synthesized using a DNA/RNA synthesizer “NTS T-series” (manufactured by Nihon Techno Service Co., Ltd.). The standard protocol for phosphoramidite-based coupling methods was used for the synthesis.

Each oligonucleotide probe carrier obtained by the automatic synthesis was reacted with ammonium water at room temperature (25° C.) for 2 hours to form a particulate carrier. The solvent of the particulate carrier was dried by a centrifugal dryer, and ultrapure water was added thereto to obtain first components 1 to 16 containing respective oligonucleotide probes 1 to 16.

The oligonucleotide probes 1 to 15 were confirmed to emit fluorescence and excimer emission by a specific excitation light (light having a wavelength of 350 nm). Note that the oligonucleotide probe 16 emits neither fluorescence nor excimer emission. The ratio of the β-form of deoxyribose in Table V was obtained as {(the number of β-deoxyriboses in an oligonucleotide probe)/(the number of deoxyriboses in the oligonucleotide probe)}×100 [%]. A “−” in Table V indicates that the oligonucleotide probe 16 does not contain the β-form of deoxyribose.

TABLE V
[Table 5]

| LUMINESCENT | | | | | | | | | | | | | | | | | |
|---|---|---|---|---|---|---|---|---|---|---|---|---|---|---|---|---|---|
| DYE | | | | | | | | | | | | | | | | | |
| MOLECULES | | 1 | 2 | 3 | 4 | 5 | 6 | 7 | 8 | 9 | 10 | 11 | 12 | 13 | 14 | 15 | 16 |
| MONOMER | 5′ | 1 | 1 | 1 | 1 | 1 | 1 | 1 | 1 | 2 | 2 | 2 | 2 | 2 | 2 | 2 | 2 |
| SEQUENCE | | 1 | 1 | 1 | 1 | 2 | 2 | 2 | 2 | 1 | 1 | 1 | 1 | 2 | 2 | 2 | 2 |
| | | 1 | 1 | 2 | 2 | 1 | 1 | 2 | 2 | 1 | 1 | 2 | 2 | 1 | 1 | 2 | 2 |
| | 3′ | 1 | 2 | 1 | 2 | 1 | 2 | 1 | 2 | 1 | 2 | 1 | 2 | 1 | 2 | 1 | 2 |

| | | | | | | | | | | | | | | | | |
|---|---|---|---|---|---|---|---|---|---|---|---|---|---|---|---|---|
| RATIO OF | 100 | 100 | 100 | 100 | 100 | 100 | 100 | 100 | 100 | 100 | 100 | 100 | 100 | 100 | 100 | — |
| β-FORM OF | | | | | | | | | | | | | | | | |
| DEOXYRIBOSE | | | | | | | | | | | | | | | | |
| (%) | | | | | | | | | | | | | | | | |
| NUMBER OF | 4 | 4 | 4 | 2 | 3 | 2 | 2 | 1 | 3 | 2 | 2 | 1 | 1 | 1 | 1 | 0 |
| CHROMOPHORES | | | | | | | | | | | | | | | | |
| OR | | | | | | | | | | | | | | | | |
| LUMINOPHORES | | | | | | | | | | | | | | | | |
| RATIO OF | 0 | 0 | 0 | 0 | 0 | 0 | 0 | 0 | 0 | 0 | 0 | 0 | 0 | 0 | 0 | 0 |
| NATURAL BASE | | | | | | | | | | | | | | | | |
| (%) | | | | | | | | | | | | | | | | |

### (2) Signal Measurement

### (2.1) Oligonucleotide Probe Placement Step

A 96-well microplate was prepared in which wells with an opening diameter of 7 mm were arranged in 12 columns×8 rows at intervals of 9 mm. Using an automatic dispenser “NichiMart CUBE” (manufactured by Nichiryo Co., Ltd.), 100 μL of each of the oligonucleotide probes 1 to 16 was placed in the 96-well microplate. Thus, a plurality of reaction fields was formed.

### (2.2) First Signal Information Acquisition Step

Fluorescence spectra obtained by irradiating the 96-well microplate on which the oligonucleotide probes were placed with excitation light having a wavelength of 350 nm were acquired as the first signal information.

### (2.3) Third Sample Placement Step

In acquisition of information only on the metabolites, 20 μL of the third sample was placed in each well of the 96-well microplate after the above first signal information acquisition step in the same manner as the oligonucleotide probes. That is, a mixture of one of the oligonucleotide probes and the third sample was placed in each well.

In acquisition of information only on the compositions constituting the cells, the fourth sample was placed instead of the third sample above.

When both the acquisition of information on the metabolites and the acquisition of information on the compositions constituting the cells were performed at the same time, instead of the third sample above, the third sample and the fourth sample were mixed at a ratio of 9:1 and placed so that the total volume was 20 μL in each well.

### (2.4) Second Signal Information Acquisition Step

Fluorescence spectra obtained by irradiating the 96-well microplate on which the third sample was placed with excitation light having a wavelength of 350 nm were acquired as the second signal information.

### (2.5) Analysis Step

The analysis data was calculated by subtracting the first signal information acquired in the first signal information acquisition step from the second signal information acquired in the second signal information acquisition step. Then, using the analysis data and the fluorescent staining measurement data as explanatory variables, a discriminant model was created by linear discriminant analysis (LDA). Thereafter, calculation of a correct answer rate and creation of a confusion matrix were performed by sixfold cross validation, and the generalization performance of the discriminant model was quantified.

The first sample was prepared in six types, Type1 to Type6, as described above. Then, for each type, 10 of the second and third samples were prepared and analyzed. That is, the analysis was performed 10 times for one type of the first sample. The number of specimens was 60.

### 6. Evaluation

Table VI shows, for the present inventions 1 to 4 and comparative examples 1 to 3, the method for acquiring the protein information (type of probe), the composition of the sample for exhaustive signal analysis, the method for acquiring information for the exhaustive signal analysis, and the accuracy of discrimination as an evaluation result. An accuracy of 70% or more was considered acceptable.

“No measurement” in the comparative examples 1 and 2 indicates that a probe was not used in the acquisition of the information for the exhaustive signal analysis. Furthermore, “no measurement” in the comparative example 3 indicates that a probe was not used in the acquisition of the information on the specific protein.

TABLE VI

| | | | METHOD FOR | |
|---|---|---|---|---|
| | METHOD FOR | | ACQUIRING | |
| | ACQUIRING | COMPOSITION OF | INFORMATION FOR | AVERAGE VALUE |
| | INFORMATION ON | SAMPLE FOR | EXHAUSTIVE | OF ACCURACY |
| | SPECIFIC PROTEIN | EXHAUSTIVE SIGNAL | SIGNAL ANALYSIS | OF |
| | (TYPE OF PROBE) | ANALYSIS | (TYPE OF PROBE) | DISCRIMINATION |
| PRESENT | HIGH-BRIGHTNESS | METABOLITES | OLIGONUCLEOTIDE | 87% |
| INVENTION | FLUORESCENT | (THIRD SAMPLE) | PROBE | |
| 1 | NANOPARTICLES | | | |
| PRESENT | HIGH-BRIGHTNESS | COMPOSITIONS | OLIGONUCLEOTIDE | 83% |
| INVENTION | FLUORESCENT | CONSTITUTING CELLS | PROBE | |
| 2 | NANOPARTICLES | (FOURTH SAMPLE) | | |
| PRESENT | HIGH-BRIGHTNESS | METABOLITES + | OLIGONUCLEOTIDE | 93% |
| INVENTION | FLUORESCENT | COMPOSITIONS | PROBE | |
| 3 | NANOPARTICLES | CONSTITUTING CELLS | | |
| | | (THIRD SAMPLE + | | |
| | | FOURTH SAMPLE) | | |
| PRESENT | FLUORESCENT DYE | METABOLITES | OLIGONUCLEOTIDE | 72% |
| INVENTION | | (THIRD SAMPLE) | PROBE | |
| 4 | | | | |
| COMPARATIVE | FLUORESCENT DYE | METABOLITES | NO MEASUREMENT | 23% |
| EXAMPLE | | (THIRD SAMPLE) | | |
| 1 | | | | |
| COMPARATIVE | HIGH-BRIGHTNESS | METABOLITES | NO MEASUREMENT | 45% |
| EXAMPLE | FLUORESCENT | (THIRD SAMPLE) | | |
| 2 | NANOPARTICLES | | | |
| COMPARATIVE | NO MEASUREMENT | METABOLITES | OLIGONUCLEOTIDE | 63% |
| EXAMPLE | | (THIRD SAMPLE) | PROBE | |
| 3 | | | | |

The generalization performance of the above discriminant model was quantified, and the accuracy of discrimination was calculated by the following method.

In the confusion matrix, row elements indicate the number of specimens for the known cell states (Type1 to Type6) and column elements indicate the number of specimens for the cell states (Type1 to Type6) based on the analysis results.

Tables VII to XIII show the respective confusion matrices for the present inventions 1 to 4 and the comparative examples 1 to 3.

TABLE VII
[Table 7]
predict label
PRESENT INVENTION 1: ACCURACY 87%

| | | Type1 | Type2 | Type3 | Type4 | Type5 | Type6 |
|---|---|---|---|---|---|---|---|
| True | Type1 | 8 | 0 | 0 | 2 | 0 | 0 |
| label | Type2 | 0 | 9 | 1 | 0 | 0 | 0 |
| | Type3 | 2 | 1 | 7 | 0 | 0 | 0 |
| | Type4 | 0 | 0 | 2 | 8 | 0 | 0 |
| | Type5 | 0 | 0 | 0 | 0 | 10 | 0 |
| | Type6 | 0 | 0 | 0 | 0 | 0 | 10 |

Table VII will be described.

For example, for the components in the row of Type1, the column element of Type1 is “8”, the column elements of Type2, Type3, Type5 and Type6 are “0”, and the column element of Type4 is “2”. That is, it is indicated that, for the 10 known specimens in which the state of the cells is Type1, eight specimens were discriminated to be Type1 and two specimens were discriminated to be Type4 based on the analysis results.

For Type1 to Type6, the number of specimens correctly discriminated was calculated. For example, in the case of Type1, the number of specimens correctly discriminated is the component (component in a bold square) that corresponds to both the row element of Type1 and the column element of Type1. For Type1 to Type6, the sum of the numbers of specimens correctly discriminated was calculated. Specifically, the sum of the components in the bold squares was 8+9+7+8+10+10=52, and the number of specimens correctly discriminated was 52. The accuracy is represented by a ratio of the number of correctly discriminated specimens to the total of 60 specimens (the number of specimens is 60). For the present invention 1, the accuracy was 87%.

TABLE VIII
[Table 8]
predict label
PRESENT INVENTION 2: ACCURACY 83%

| | | Type1 | Type2 | Type3 | Type4 | Type5 | Type6 |
|---|---|---|---|---|---|---|---|
| True | Type1 | 8 | 0 | 0 | 2 | 0 | 0 |
| label | Type2 | 0 | 9 | 1 | 0 | 0 | 0 |
| | Type3 | 2 | 1 | 7 | 0 | 0 | 0 |
| | Type4 | 0 | 0 | 2 | 8 | 0 | 0 |
| | Type5 | 0 | 0 | 0 | 0 | 9 | 1 |
| | Type6 | 0 | 0 | 0 | 0 | 1 | 9 |

TABLE IX
[Table 9]
predict label
PRESENT INVENTION 3: ACCURACY 93%

| | | Type1 | Type2 | Type3 | Type4 | Type5 | Type6 |
|---|---|---|---|---|---|---|---|
| True | Type1 | 10 | 0 | 0 | 0 | 0 | 0 |
| label | Type2 | 0 | 10 | 0 | 0 | 0 | 0 |
| | Type3 | 0 | 0 | 8 | 2 | 0 | 0 |
| | Type4 | 0 | 0 | 2 | 8 | 0 | 0 |
| | Type5 | 0 | 0 | 0 | 0 | 10 | 0 |
| | Type6 | 0 | 0 | 0 | 0 | 0 | 10 |

TABLE X
[Table 10]
predict label
PRESENT INVENTION 4: ACCURACY 72%

| | | Type1 | 1 Type2 | Type3 | Type4 | Type5 | Type6 |
|---|---|---|---|---|---|---|---|
| True | Type1 | 7 | 0 | 1 | 0 | 0 | 2 |
| label | Type2 | 3 | 7 | 0 | 0 | 0 | 0 |
| | Type3 | 0 | 1 | 6 | 3 | 0 | 0 |
| | Type4 | 0 | 2 | 1 | 7 | 0 | 0 |
| | Type5 | 0 | 0 | 2 | 0 | 8 | 0 |
| | Type6 | 0 | 0 | 0 | 0 | 2 | 8 |

TABLE XI
[Table 11]
predict label
COMPARATIVE EXAMPLE 1: ACCURACY 23%

| | | Type1 | Type2 | Type3 | Type4 | Type5 | Type6 |
|---|---|---|---|---|---|---|---|
| True | Type1 | 4 | 0 | 4 | 0 | 0 | 2 |
| label | Type2 | 1 | 2 | 1 | 0 | 4 | 2 |
| | Type3 | 0 | 4 | 0 | 4 | 1 | 1 |
| | Type4 | 0 | 2 | 3 | 4 | 0 | 1 |
| | Type5 | 2 | 2 | 2 | 2 | 1 | 1 |
| | Type6 | 3 | 0 | 0 | 0 | 4 | 3 |

TABLE XII
[Table 12]
predict label
COMPARATIVE EXAMPLE 2: ACCURACY 45%

| | | Type1 | Type2 | Type3 | Type4 | Type5 | Type6 |
|---|---|---|---|---|---|---|---|
| True | Type1 | 4 | 2 | 3 | 0 | 0 | 1 |
| label | Type2 | 1 | 5 | 0 | 0 | 2 | 2 |
| | Type3 | 2 | 2 | 3 | 2 | 0 | 1 |
| | Type4 | 0 | 1 | 2 | 6 | 0 | 1 |
| | Type5 | 0 | 0 | 2 | 2 | 5 | 1 |
| | Type6 | 3 | 0 | 0 | 0 | 3 | 4 |

TABLE XIII
[Table 13]
predict label
COMPARATIVE EXAMPLE 3: ACCURACY 63%

| | | Type1 | Type2 | Type3 | Type4 | Type5 | Type6 |
|---|---|---|---|---|---|---|---|
| True | Type1 | 8 | 1 | 0 | 1 | 0 | 0 |
| label | Type2 | 1 | 6 | 1 | 1 | 0 | 1 |
| | Type3 | 0 | 1 | 8 | 0 | 1 | 0 |
| | Type4 | 0 | 1 | 0 | 6 | 0 | 3 |
| | Type5 | 1 | 1 | 1 | 1 | 5 | 1 |
| | Type6 | 1 | 0 | 0 | 1 | 4 | 5 |

In the present invention 2, the number of correctly discriminated specimens was 50, and the accuracy was 83%.

In the present invention 3, the number of correctly discriminated specimens was 56, and the accuracy was 93%.

In the present invention 4, the number of correctly discriminated specimens was 43, and the accuracy was 72%.

In the comparative example 1, the number of correctly discriminated specimens was 14, and the accuracy was 23%.

In the comparative example 2, the number of correctly discriminated specimens was 27, and the accuracy was 45%.

In the comparative example 3, the number of correctly discriminated specimens was 38, and the accuracy was 63%.

From the present Examples, it can be seen that the analysis method according to the present invention can improve the accuracy.

From a comparison between the present inventions 1 and 4, it can be seen that the accuracy is further improved by using the high-brightness fluorescent nanoparticles as a method for acquiring protein information.

### INDUSTRIAL APPLICABILITY

By using the present invention, the accuracy of analysis can be improved in the culture sensing technology. As a result, in a production system of cultured cells, the culture process can be stabilized and the process efficiency can be improved, so that productivity can be improved.

### REFERENCE SIGNS LIST

S101 first component placement step
S102 first signal information acquisition step
S103 second component placement step
S104 second signal acquisition step
S105 analysis step

1. An analysis method comprising the steps of:
acquiring first information on expression of a specific protein that characterizes a function of a cell or a microorganism;
acquiring second information on at least one of:
a metabolite produced by the cell or the microorganism as a result of performing the function; or
a composition constituting the cell or the microorganism; and,
based on the first information and the second information, analyzing at least the function or a state of the cell or the microorganism.

2. The analysis method according to claim 1, further comprising:
separating, from a first sample obtained by culturing the cell or the microorganism in a culture solution, the cultured cell or microorganism and a rest of the culture solution to respectively obtain a second sample and a third sample;
crushing the cultured cell or microorganism to obtain a fourth sample;
acquiring, from the second sample, the first information on the expression of the specific protein that characterizes the function of the cell or the microorganism;
acquiring, from the third sample or the fourth sample, the second information on at least one of:
the metabolite produced by the cell or the microorganism as a result of performing the function; or
the composition constituting the cell or the microorganism; and,
based on the first information and the second information, analyzing at least the function and the state of the cell or the microorganism.

3. The analysis method according to claim 1, wherein at least one of the information on the expression of the protein, the information on the metabolite, or the information on the composition constituting the cell or the microorganism is obtained using a probe labeled with a luminescent substance.

4. The analysis method according to claim 3, wherein the probe labeled with the luminescent substance is a probe that includes a resin particle containing a luminescent dye or is an oligonucleotide probe.

5. The analysis method according to claim 4, wherein
the acquiring the first information on the expression of the protein includes using the probe that includes the resin particle containing the luminescent dye, and
the acquiring the second information on at least one of:
the metabolite; or the composition constituting the cell or
the microorganism
includes using the oligonucleotide probe labeled with the luminescent substance.

6. An analysis system for the cell or the microorganism, using the analysis method according to claim 1.
